(19) United States
(12) Patent Application Publication
(10) Pub. No.: US 2004/0151758 A1
(43) Pub. Date: Aug. 5, 2004

(54) Fat substitutes

(76) Inventors: William J. Wechter, Ojai, CA (US); Elaine Benaksas Schwartz, Yorba Linda, CA (US); E. David Murray, Redlands, CA (US); Israel Gutierrez, Colton, CA (US)

Correspondence Address: PETERS, VERNY, JONES & SCHMITT LLP, Suite 6, 385 Sherman Avenue, Palo Alto, CA, 94306, US

(21) Appl. No.: 10/763,111

(22) Filed: Jan. 21, 2004

Related U.S. Application Data
(60) Provisional application No. 60/441,892, filed on Jan. 22, 2003.

(51) Int. Cl. A61K 47/00

(52) U.S. Cl. 424/439

(57) ABSTRACT

The present invention concerns fat substitutes that are compounds, which are fatty acids whose yield energy from beta-oxidation is sufficiently low so that the compound is rendered metabolically low caloric or non-caloric, or a physiologically acceptable ester thereof or a metabolic precursor thereof. The compounds of the invention may be used as fat substitutes, either alone or in combination with other fats or in food compositions, and the like.

### CROSS-REFERENCE TO RELATED APPLICATIONS

This application claims the benefit of the subject matter disclosed in prior copending Provisional Patent Application Serial No. 60/441,892 filed Jan. 22, 2003, the disclosure of which is incorporated herein by reference in its entirety.

### BACKGROUND OF THE INVENTION

The present invention relates to edible fat materials that yield low or no caloric energy on consumption. More particularly, the present invention relates to compounds and compositions that may be employed as fat substitutes as well as to food compositions employing these fat substitutes.

The prevalence of obesity is on the rise and health care costs directly attributed to obesity are in the tens of billions of dollars and additional tens of billions per year are spent on weight reduction programs and special foods.

Obesity is associated with increased morbidity and mortality. It has been linked to a number of diseases including type 2 diabetes mellitus, hypertension, coronary artery disease, stroke, hypercholesterolemia, cholelithiasis, fatty liver disease, certain cancers (postmenopausal breast cancer and cancers of the colon, endometrium and kidney), musculoskeletal disorders (osteoarthritis), obstructive sleep apnea, and infertility, not to mention the social consequences and isolation that many patients with obesity experience.

Obesity is perceived as a common problem in contemporary society. This condition is due, in many people, to a greater intake of calories than are expended. While genetic and behavioral factors play a significant role, it is generally agreed that reasonable modifications of the caloric value of foods can be valuable in reaching a more desirable equilibrium weight for an individual predisposed to obesity.

Many foods that consumers find pleasing to the taste contain significant fat levels. This can be a problem for individuals drawn to these foods because fat has about twice the caloric density of protein and carbohydrates. It has, in fact, been estimated that fat contributes about 40% of the total calories in the diet. It has long been desired to reduce the available calories of dietary fat without decreasing the appeal or taste expected of fatty foods.

Unfortunately, of the materials previously suggested as fat replacements, few have all of the desirable attributes of natural triglyceride fats and oils. One approach to lower the caloric value of edible fat has been to decrease the amount of triglyceride that is absorbed in the human system since the usual edible triglyceride fats are almost completely absorbed. U.S. Pat. No. 2,962,419, to Minich discloses that fatty acid esters, which contain a neopentyl nucleus, are not digested like normal fats and thus can be used as fat substitutes in food compositions.

Several other patents disclose edible compounds that are not digested or absorbed to the same extent as natural triglycerides. In U.S. Pat. No. 3,579,548, White discloses certain glycerol esters of certain branched carboxylic acids, which are said to have these properties. In U.S. Pat. No. 3,600,186, Mattson and Volpenhein disclose sugar and sugar alcohol fatty acid esters having at least four fatty acid ester groups. All of these compounds are said to possess physical properties similar to ordinary triglyceride fat, but are absorbed less readily when eaten.

In U.S. Pat. No. 4,508,746, Hamm discloses a low-calorie substitute for at least a portion of the edible oil component in oil-based food compositions. The low-calorie substitute is comprised in substantial proportion of at least one low-calorie oil component selected from the group consisting of thermally stable polycarboxylic acids having 2 to 4 carboxylic acid groups esterified with saturated or unsaturated alcohols having straight or branched carbon chains from 8 to 30 carbon atoms.

In another attempt at simulating the natural properties of fat, Fulcher discloses certain diesters in U.S. Pat. No. 4,582,927. These compounds have at least two carboxylate groups joined to a common carbon atom, with each of the carboxylate groups containing the residue of a 12 to 18 carbon alkyl, alkenyl, or dienyl alcohol.

There remains a need, however, for a fat substitute that has good absorbability. The fat substitute should mimic conventional triglyceride fat by affording the same utility in various fat-containing food compositions such as shortening, margarine, cake mixes, and the like, and be useful in frying or baking. It is also desirable that a fat substitute not leach fat-soluble vitamins and minerals from the body nor function as purgatives. The fat substitutes should be compatible with the digestive processes of humans and other mammals.

### SUMMARY OF THE INVENTION

One embodiment of the present invention is a method of reducing or eliminating metabolic caloric content of a food composition comprising an edible fat. The method comprises substituting, for at least a portion of the edible fat of the food composition, a compound that is a fatty acid whose yield energy from beta-oxidation is sufficiently low that the compound is rendered metabolically low caloric or non-caloric, or a physiologically acceptable ester thereof or a metabolic precursor thereof.

Another embodiment of the present invention is a method for rendering an organic acid compound metabolically low caloric or non-caloric and the organic acid compounds produced. In one embodiment the method comprises introducing into the compound one or more substituents at a carbon atom that is alpha to a carboxyl group of the organic acid compound that renders the compound incapable of undergoing beta-oxidation. In this embodiment the substituents render such carbon atom quaternary such that the carbon atom no longer comprises a hydrogen moiety or, where such carbon atom does comprise a hydrogen moiety, render such carbon atom a chiral center having a predominantly S-stereochemical configuration or an S-configuration substantially free from R-configuration. In another embodiment one or more substituents are introduced into the organic acid compound such that chiral centers are produced or at least one quaternary carbon atom is produced. One or more of these chiral centers or quaternary carbon atoms become beta to a carboxylic acid functionality that is formed as a result of metabolic degradation. Alpha-oxidation then makes the chiral center alpha to the free carboxylic acid produced by the alpha-oxidation. If the chiral center has a predominantly S-configuration or an S-configuration substantially free from R-configuration or if the carbon atom is quaternary, a CoA ester cannot be made. If the chiral center has predominantly or exclusively an R-configuration, the CoA ester can be made using ATP, thus using energy. Thus, an R-configuration for the chiral center uses energy (thereby rendering the organic acid compound low caloric) and an S-configuration for the chiral center or a quaternary carbon atom stops the oxidation process (thereby rendering the organic acid compound non-caloric if no beta oxidation has occurred or low caloric if some beta oxidation has occurred). The substituent(s) renders the yield energy from beta-oxidation of the organic acid compound sufficiently low that the compound is rendered metabolically low caloric or non-caloric.

### BRIEF DESCRIPTION OF THE DRAWING

FIG. 1A is a schematic depicting an example of a reaction scheme for the preparation of compounds for use in accordance with the present invention.

FIG. 1B is a schematic depicting an example of a reaction scheme for the preparation of compounds for use in accordance with the present invention.

### DETAILED DESCRIPTION OF SPECIFIC EMBODIMENTS OF THE INVENTION

It has surprisingly been discovered that the number of substituents on the alpha carbon of a fatty acid, and in some instances, the stereoisomeric arrangement of substituents on the alpha carbon of a fatty acid and/or the stereoisomeric arrangement of other chiral centers present in a fatty acid, renders such fatty acid and physiologically acceptable esters thereof useful as fat substitutes that are metabolically low caloric or non-caloric. In some embodiments, the number of substituents or the stereoisomeric arrangement of substituents renders the fatty acid incapable of beta-oxidation. In this case, there is no energy obtained (“yield energy”) from beta-oxidation of the fatty acid compound since no beta-oxidation occurs to any significant extent. Such fatty acid compounds are metabolically non-caloric and may be used to substitute for at least a portion or all of the edible fat in a prepared food composition. In other instances, the stereoisomeric arrangement of substituents on the alpha carbon renders the yield energy from beta-oxidation of the fatty acid sufficiently low so that the fatty acid compound is rendered metabolically low caloric.

The aforementioned metabolic characteristics may be realized by the presence in the fatty acid of a “metabolic blocker,” which is any moiety that renders the fatty acid non-caloric or low caloric. This is achieved by rendering the fatty acid incapable of beta-oxidation or, if capable of beta-oxidation, by directing the fatty acid to undergo alpha-oxidation after undergoing beta-oxidation thereby using energy. One example of a metabolic blocker is a quaternary carbon atom, that is, a carbon atom that does not have a hydrogen substituent. The quaternary carbon atom may be alpha to the carboxylic acid functionality in which case the fatty acid does not undergo beta-oxidation and, thus, the fatty acid is rendered non-caloric by the metabolic blocker. In another embodiment the quaternary carbon atom is at a position in the fatty acid other than alpha to the carboxylic acid functionality in which case some oxidation of the fatty acid may occur until a carboxylic acid functionality is created during oxidation such that the quaternary carbon atom becomes alpha to the carboxylic acid functionality. In this latter embodiment, the fatty acid is rendered low caloric since some oxidation may occur until the quaternary carbon atom is able to prevent beta-oxidation.

In another embodiment the fatty acid comprises a chiral center that has a single hydrogen substituent and the stereochemical configuration of the chiral center is S. Such a chiral center may be alpha to the carboxylic acid functionality. In this case, if the chiral center is an S configuration substantially free from an R configuration, the fatty acid is rendered substantially incapable of undergoing beta-oxidation thereby rendering the fatty acid substantially non-caloric. However, in the above situation, the R stereoisomer may be present with the S stereoisomer, and the compound is rendered low caloric. The degree of the low caloric character is dependent on the amount of S stereoisomer versus the amount of R stereoisomer, namely, the more of the S stereoisomer that is present, the lower the caloric character of the compound.

In another embodiment the chiral center having the S configuration is at a position in the fatty acid other than alpha to the carboxylic acid functionality in which case some oxidation of the fatty acid may occur until a free carboxylic acid functionality is created during oxidation (alpha or beta) such that the chiral center having the S configuration becomes alpha to the carboxylic acid functionality. In this latter embodiment, the fatty acid is rendered low caloric since some oxidation may occur until the chiral center having the S configuration is able to prevent beta-oxidation. In this latter situation there may be chiral centers, which are present in the carbon chain of the fatty acid between the chiral center having the S configuration and the carboxylic acid functionality, that have an R stereochemical configuration. When these chiral centers are beta to the carboxylic acid functionality, alpha-oxidation of the fatty acid occurs. When alpha-oxidation occurs, it results in a free carboxylic acid with the chiral center alpha to the carboxylic acid. If the chiral center is R, energy is used to form the CoA ester; if the chiral center is S the CoA ester cannot be formed and the fatty acid can no longer yield calories.

As mentioned above, the metabolic blocker may be a plurality of chiral centers in a carbon chain of the fatty acid where the chiral centers have a predetermined stereochemical configuration. On the other hand, the metabolic blocker may comprise a plurality of chiral centers in a carbon chain of the fatty acid where one or more of the chiral centers are spaced apart by a designated number of carbon atoms such that beta-oxidation is avoided and alpha-oxidation is promoted thereby rendering the fatty acid low caloric. The stereochemical configuration of the chiral centers is R in many embodiments. To render the fatty acid even more low caloric the stereochemical configuration of the first chiral center encountered after an alpha-oxidation process is S causing oxidation to stop after the alpha-oxidation process, which is essentially as described above. In this embodiment, the number of carbon atoms in between the chiral centers is determined by the need for alpha-oxidation to occur by having the chiral center end up beta to the carboxylic acid as a result of oxidation processes. This number is generally 2 or 4 and so forth.

By the term “alpha-oxidation” is meant that a carbon atom alpha to a CoA ester of a carboxylic acid moiety is oxidized to form a free carboxylic acid moiety. Subsequent formation of a CoA ester of the free carboxylic acid moiety uses energy.

By the term “beta-oxidation” is meant that a carbon atom beta to a CoA ester of a carboxylic acid moiety is oxidized to form a CoA ester of a carboxylic acid moiety.

As mentioned above, an embodiment of the present invention is a method of reducing or eliminating metabolic caloric content of a food composition comprising an edible fat. The method comprises substituting, for at least a portion of the edible fat of the food composition, a compound that is a fatty acid whose yield energy from beta-oxidation is sufficiently low that the compound is rendered metabolically low caloric or non-caloric, or a physiologically acceptable ester thereof or a metabolic precursor thereof.

In addition, as mentioned above, another embodiment of the present invention is a method for rendering an organic acid compound metabolically low caloric or non-caloric and another embodiment is the organic acid compounds produced. In one embodiment the method comprises introducing into the compound one or more substituents at a carbon atom that is alpha to a carboxyl group of the organic acid compound that renders the compound incapable of undergoing beta-oxidation. In this embodiment the substituents render such carbon atom quaternary such that the carbon atom no longer comprises a hydrogen moiety or, where such carbon atom does comprise a hydrogen moiety, render such carbon atom a chiral center having an S-stereochemical configuration. In another embodiment one or more substituents are introduced into the organic acid compound such that chiral centers are produced or at least one quaternary carbon atom is produced. One or more of these chiral centers or quaternary carbon atoms become beta to a carboxylic acid functionality that is formed as a result of metabolic degradation. Alpha-oxidation then makes the chiral center alpha to the free carboxylic acid produced by the alpha-oxidation. If the chiral center has an S-configuration or if the carbon atom is quaternary, a CoA ester cannot be made. If the chiral center has an R-configuration, the CoA ester can be made using ATP, thus using energy. Thus, an R-configuration for the chiral center uses energy (thereby rendering the organic acid compound low caloric) and an S-configuration for the chiral center or a quaternary carbon atom stops the oxidation process (thereby rendering the organic acid compound non-caloric if no beta oxidation has occurred or low caloric if some beta oxidation has occurred). The substituent(s) renders the yield energy from beta-oxidation of the organic acid compound sufficiently low that the compound is rendered metabolically low caloric or non-caloric.

In one specific embodiment the alpha carbon does contain a single hydrogen substituent rendering the alpha carbon a chiral center, in which case the stereoisomeric form of the fatty acid is the S-enantiomer substantially free from the R-enantiomer or the S-enantiomer that is present is at least at about 50% of an enantiomeric mixture. The S-enantiomer of such a fatty acid does not undergo beta-oxidation to any significant extent and, thus, there is substantially no yield energy from the beta-oxidation of such a fatty acid. Although it is not required to propose a mechanism by which the invention is thought to work and we do not want to be bound by any particular mechanism, it is believed that the S-enantiomer of the fatty acid does not react with Fatty Acid CoA ligase in vivo, which is a necessary step for beta-oxidation of the fatty acid. The fatty acid is rendered substantially unmetabolizable or non-metabolizable and may be used to substitute for at least a portion or all of the edible fat in a prepared food composition.

The degree of caloric character of the compound may be adjusted so that the compound is reduced caloric or non-caloric. The degree is dependent on the amount of S stereoisomer versus the amount of R stereoisomer, namely, the more of the S stereoisomer that is present, the lower the caloric character of the compound and, if the S stereoisomer is substantially free from R stereoisomer, the compound is rendered non-caloric.

In another specific embodiment the fatty acid compound comprises a single hydrogen substituent at the alpha carbon (carbon 1 not counting the carboxyl carbon) and at carbon 5, and, alternatively, also at carbon 9, and further alternatively, also at carbon 13, and further alternatively, also at carbon 17, and further alternatively, also at carbon 21, and further alternatively, also at carbon 24, and so forth. Accordingly, the aforementioned carbon atoms of the fatty acid compound represent chiral centers. In this situation, the fatty acid compound may comprise R-enantiomers at each of the chiral centers. Although the R-enantiomer at carbon 1 (alpha carbon) does undergo beta-oxidation, the yield energy from such beta-oxidation is sufficiently low enough to render the fatty acid compound low caloric and useful as a fat substitute. This results because of the presence of multiple chiral centers. Again, not wanting to be held to any particular theory or mechanism, it is believed that the synthesis of the CoA ester in vivo resulting from the action of CoA ligase on the R-enantiomeric centers consumes much of the ATP generated during beta-oxidation. Accordingly, the yield energy from the beta-oxidation is substantially reduced and may be used to substitute for at least a portion or all of the edible fat in a prepared food composition. It should be pointed out that in the aforementioned embodiment S-enantiomers may be present since, as explained above, the S enantiomers are substantially incapable of formation of CoA esters. This embodiment is exemplified by phytanic acid. For phytanic acid, once the initial alpha oxidation occurs the resulting pristanic acid either enters beta-oxidation with no further energy expenditure if the alpha chiral center is R (no more alpha oxidation occurs giving rise to the free carboxylic acid that uses energy to be esterified with CoA again). On the other hand, if the alpha chiral center is S, no other oxidation occurs since the CoA is not formed.

In another specific embodiment the fatty acid compound comprises a single hydrogen substituent at the alpha carbon (carbon 1 not counting the carboxyl carbon) and at carbon 4, and, alternatively, also at carbon 7, and further alternatively, also at carbon 10, and further alternatively, also at carbon 13, and further alternatively, also at carbon 16, and further alternatively, also at carbon 19, and so forth. Accordingly, the aforementioned carbon atoms of the fatty acid compound represent chiral centers. In this situation, the fatty acid compound may comprise R-enantiomers at each of the chiral centers. Although the R-enantiomer at carbon 1 (alpha carbon) does undergo beta-oxidation, the yield energy from such beta-oxidation is sufficiently low enough to render the fatty acid compound low caloric and useful as a fat substitute. This results because of the presence of multiple chiral centers that in this circumstance have two methylene (—CH₂—) groups between them. In this situation, after one round of beta-oxidation the fatty acid compound must undergo alpha-oxidation to permit the next round of beta-oxidation. The alpha-oxidation results in a free carboxyl group as opposed to beta-oxidation, which results in a CoA ester for subsequent oxidation steps. This free carboxyl must be re-esterified to a CoA ester. Again, not wanting to be held to any particular theory or mechanism, it is believed that the synthesis of the CoA ester in vivo resulting from the action of CoA ligase on the R-enantiomeric centers consumes much of the ATP generated during beta-oxidation. Accordingly, the yield energy from the beta-oxidation and alpha-oxidation is substantially reduced and may be used to substitute for at least a portion or all of the edible fat in a prepared food composition. It should be pointed out that in the aforementioned embodiment S-enantiomers may be present since, as explained above, the S enantiomers are substantially incapable of formation of CoA esters.

In one embodiment the compound employed as the fat substitute has the formula:

A-(metabolic blocker)-CO₂H

wherein the metabolic blocker renders the yield energy from beta-oxidation of the compound sufficiently low that the compound is rendered metabolically low caloric or non-caloric and wherein A is alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations, or alkyne having from 2 to about 30 carbon atoms and having from 1 to about 10 unsaturations, or substituted alkyne having 2 to about 30 carbon atoms and having from 1 to about 10 unsaturations, or a physiologically acceptable ester thereof or a metabolic precursor thereof.

In one embodiment of the above the metabolic blocker has the formula:

—C(X)(D)-,

wherein X is alkyl of 1 to about 10 carbon atoms and D is hydrogen or alkyl of 1 to about 10 carbon atoms and wherein, when D is hydrogen, the metabolic blocker is an enantiomeric mixture that comprises at least 50% of the S-enantiomer up to an S-enantiomer substantially free from R-enantiomer and wherein A is alkyl of 5 to about 30 carbon atoms, substituted alkyl of 5 to about 30 carbon atoms, alkene of 5 to about 30 carbon atoms and having from 1 to about 10 unsaturations, or substituted alkene having from 5 to about 30 carbon atoms and having from 1 to about 10 unsaturations.

In a particular embodiment of the fatty acid compounds of the above formulas, A is selected from the group consisting of CₙH₂ₙ₊₁, CₙH₂ₙ₋₁, CₙH₂ₙ₋₃, and CₙH₂ₙ₋₅ wherein n is 1 to about 20 and wherein 1 to about 5 carbon atoms are optionally substituted with alkyl, branched alkyl, alkenyl, or alkynyl groups and the like.

In one embodiment of the above the metabolic blocker has the formula:

—(CH₂(CH₂)ₐCH(Y)(CH₂(CH₂)_{b}CH(Y))ₘ(CH₂(CH₂)_{c}CH(V)—

wherein m is 0 to 3 and wherein V and Y are independently alkyl having from 1 to about 10 carbon atoms and

(i) wherein when the carbon atom comprising the V group is predominantly an S-enantiomer or is an S-enantiomer substantially free from R-enantiomer, the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and a, b and c are independently an integer of 1 to 5 or

(ii) wherein, when the carbon atom comprising the V group is predominantly an r-enantiomer or is an R-enantiomer substantially free from S-enantiomer and one of the carbon atoms comprising the Y group is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and a, b and c are independently an integer of 1 to 5 and the b or c before the carbon atom that is the S-enantiomer is an integer of 1 or 3 or

(iii) wherein, when the carbon atom comprising the V group is predominantly an R-enantiomer or is an R-enantiomer substantially free from S-enantiomer and the carbon atoms comprising the Y-groups are predominantly an R-enantiomer or is an R-enantiomer substantially free from S-enantiomer, at least one of a, b and c is an integer of 1 or 3 and the others are an integer of 1 to 5 and wherein the carbon atoms not comprising the V group or the Y groups may be substituted with one or more substituents and

wherein A is alkyl of 1 to about 20 carbon atoms, substituted alkyl of 1 to about 20 carbon atoms, alkene of 2 to about 20 carbon atoms and having from 1 to about 10 unsaturations or 1 to about 5 unsaturations or 1 to about 3 unsaturations or 1 to 2 unsaturations, or substituted alkene having from 2 to about 20 carbon atoms and having from 1 to about 10 unsaturations or 1 to about 5 unsaturations or 1 to about 3 unsaturations or 1 to 2 unsaturations.

In a particular embodiment of the above, the metabolic blocker has the formula:

—CH₂CH₂CH(Y)(CH₂CH₂CH(Y))ₘCH₂CH₂CH₂CH(V)—

wherein m, V and Y are as described above. In another particular embodiment of the above the metabolic blocker has the formula:

—CH₂CH₂CH(Y)(CH₂CH₂CH₂CH₂CH(Y))ₘCH₂CH₂CH₂CH(V)—

wherein m, V and Y are as described above.

In a particular embodiment of the fatty acid compounds of the above formulas, A is selected from the group consisting of CₙH₂ₙ₊₁CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

Another embodiment of the present invention is a compound of the formula:

A′-(metabolic blocker)-CO₂H

wherein the metabolic blocker renders the compound substantially incapable of beta-oxidation and wherein A′ is alkyl of about 4 to about 30 carbon atoms, substituted alkyl of about 4 to about 30 carbon atoms, alkene of about 4 to about 30 carbon atoms and having from 1 to about 10 unsaturations, or 1 to about 5 unsaturations or 1 to about 3 unsaturations or 1 to 2 unsaturations, or substituted alkene having from about 4 to about 30 carbon atoms and having from 1 to about 10 unsaturations, or 1 to about 5 unsaturations or 1 to about 3 unsaturations or 1 to 2 unsaturations, or alkyne having from 2 to about 30 carbon atoms and having from 1 to about 10 unsaturations, or 1 to about 5 unsaturations or 1 to about 3 unsaturations or 1 to 2 unsaturations, or substituted alkyne having 2 to about 30 carbon atoms and having from 1 to about 10 unsaturations, or 1 to about 5 unsaturations or 1 to about 3 unsaturations or 1 to 2 unsaturations, or a physiologically acceptable ester thereof or a metabolic precursor thereof.

Another embodiment of the present invention is a compound of the formula:

A″-C(X²)(D¹)-CO₂H

wherein X² is alkyl of 1 to about 10 carbon atoms and D¹ is alkyl of 1 to about 10 carbon atoms and wherein A″ is alkyl of about 6 to about 30 carbon atoms, substituted alkyl of 6 to about 30 carbon atoms, alkene of about 6 to about 30 carbon atoms and having from 1 to about 10 unsaturations, or 1 to about 5 unsaturations or 1 to about 3 unsaturations or 1 to 2 unsaturations, or substituted alkene having from about 6 to about 30 carbon atoms and having from 1 to about 10 unsaturations, or 1 to about 5 unsaturations or 1 to about 3 unsaturations or 1 to 2 unsaturations, or a physiologically acceptable ester thereof or a metabolic precursor thereof.

In a particular embodiment of the fatty acid compounds of the above formulas, A″ is selected from the group consisting of CₙH₂ₙ₊₁, CₙH₂ₙ₋₁, CₙH₂ₙ₋₃, and CₙH₂ₙ₋₅ wherein n is 1 to about 20 and wherein 1 to about 5 carbon atoms are optionally substituted with alkyl, branched alkyl, alkenyl, or alkynyl groups and the like.

Another embodiment of the present invention is a compound of the formula:

A″-(metabolic blocker)-CO₂H

wherein the metabolic blocker renders the energy requirement for beta-oxidation of the compound great enough to render the compound metabolically low caloric and wherein A″ is alkyl of 1 to about 20 carbon atoms, substituted alkyl of 1 to about 20 carbon atoms, alkene of 2 to about 20 carbon atoms and having from 1 to about 10 unsaturations, or 1 to about 5 unsaturations or 1 to about 3 unsaturations or 1 to 2 unsaturations, or substituted alkene having from 2 to about 20 carbon atoms and having from 1 to about 10 unsaturations, or 1 to about 5 unsaturations or 1 to about 3 unsaturations or 1 to 2 unsaturations, or alkyne having from 2 to about 20 carbon atoms and having from 1 to about 10 unsaturations, or 1 to about 5 unsaturations or 1 to about 3 unsaturations or 1 to 2 unsaturations, or substituted alkyne having 2 to about 20 carbon atoms and having from 1 to about 10 unsaturations, or 1 to about 5 unsaturations or 1 to about 3 unsaturations or 1 to 2 unsaturations, and wherein the metabolic blocker has the formula:

—(CH₂(CH₂)ₐCH(Y)(CH₂(CH₂)_{b}CH(Y))ₘ(CH₂(CH₂)_{c}CH(V)—

wherein m is 0 to 3 and wherein V and Y are independently alkyl having from 1 to about 10 carbon atoms and

(i) wherein when the carbon atom comprising the V group is predominantly an S-enantiomer or is an S-enantiomer substantially free from R-enantiomer, the carbon atoms comprising the Y groups are independently predominantly an R-enantiomers or are R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and a, b and c are independently an integer of 1 to 5 or

(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and a, b and c are independently an integer of 1 to 5 and the b or c before the carbon atom that is the S-enantiomer is an integer of 1 or 3 and the b or c before the carbon atom that is the S-enantiomer is an integer of 1 or 3 or

(iii) wherein, when the carbon atom comprising the V group is predominantly an R-enantiomer or is an R-enantiomer substantially free from S-enantiomer and the carbon atoms comprising the Y-groups are predominantly an R-enantiomers or are R-enantiomers substantially free from S-enantiomers, at least one of a, b and c is an integer of 1 or 3 and the others of a, b and c are an integer of 1 to 5 and wherein the carbon atoms not comprising the V group or the Y groups may be substituted with one or more substituents.

In a particular embodiment of the above the metabolic blocker has the formula:

—CH₂CH₂CH(Y)(CH₂CH₂CH(Y))ₘCH₂CH₂CH₂CH(V)—

and m, V and Y are as described above. In another particular embodiment of the above the metabolic blocker has the formula:

—CH₂CH₂CH(Y)(CH₂CH₂CH₂CH₂CH(Y))ₘCH₂CH₂CH₂CH(V)—

and m, V and Y are as described above.

In a particular embodiment of the fatty acid compounds of the above formulas, A″ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

Another embodiment of the present invention is a physiologically acceptable ester of a compound of the formula:

A-(metabolic blocker)-CO₂H

wherein the metabolic blocker renders the yield energy from beta-oxidation of the compound sufficiently low that the compound is rendered metabolically low caloric or non-caloric and wherein A is alkyl of 1 to about 30 carbon atoms, substituted alkyl of 1 to about 30 carbon atoms, alkene of 2 to about 30 carbon atoms and having from 1 to about 10 unsaturations, or 1 to about 5 unsaturations or 1 to about 3 unsaturations or 1 to 2 unsaturations, or substituted alkene having from 2 to about 30 carbon atoms and having from 1 to about 10 unsaturations, or 1 to about 5 unsaturations or 1 to about 3 unsaturations or 1 to 2 unsaturations, or alkyne having from 2 to about 30 carbon atoms, and having from 1 to about 10 unsaturations, or 1 to about 5 unsaturations or 1 to about 3 unsaturations or 1 to 2 unsaturations, or substituted alkyne having 2 to about 30 carbon atoms and having from 1 to about 10 unsaturations, or 1 to about 5 unsaturations or 1 to about 3 unsaturations or 1 to 2 unsaturations, or a physiologically acceptable ester thereof.

Another embodiment of the present invention is a physiologically acceptable ester of a compound, which is a fatty acid substantially incapable of beta-oxidation, or a physiologically acceptable ester thereof.

Another embodiment of the present invention is a physiologically acceptable ester of a compound of the formula:

A′-(metabolic blocker)-CO₂H

wherein the metabolic blocker renders the compound substantially incapable of beta-oxidation and wherein A′ is alkyl of about 4 to about 30 carbon atoms, substituted alkyl of about 4 to about 30 carbon atoms, alkene of about 4 to about 30 carbon atoms and having from 1 to about 10 unsaturations, or 1 to about 5 unsaturations or 1 to about 3 unsaturations or 1 to 2 unsaturations, or substituted alkene having from about 4 to about 30 carbon atoms and having from 1 to about 10 unsaturations, or 1 to about 5 unsaturations or 1 to about 3 unsaturations or 1 to 2 unsaturations, or alkyne having from 4 to about 30 carbon atoms, and having from 1 to about 10 unsaturations, or 1 to about 5 unsaturations or 1 to about 3 unsaturations or 1 to 2 unsaturations, or substituted alkyne having 4 to about 30 carbon atoms and having from 1 to about 10 unsaturations, or 1 to about 5 unsaturations or 1 to about 3 unsaturations or 1 to 2 unsaturations, or a metabolic precursor thereof.

Another embodiment of the present invention is a physiologically acceptable ester of a compound, which is a fatty acid having a yield energy from beta-oxidation that is low enough to render the fatty acid metabolically low caloric, or a metabolic precursor thereof.

Another embodiment of the present invention is a physiologically acceptable ester of a compound of the formula:

A″-(metabolic blocker)-CO₂H

wherein the metabolic blocker renders the energy requirement for beta-oxidation of the compound great enough to render the compound metabolically low caloric and wherein A″ is alkyl of 1 to about 20 carbon atoms, substituted alkyl of 1 to about 20 carbon atoms, alkene of 2 to about 20 carbon atoms and having from 1 to about 10 unsaturations, or 1 to about 5 unsaturations or 1 to about 3 unsaturations or 1 to 2 unsaturations, or substituted alkene having from 2 to about 20 carbon atoms and having from 1 to about 10 unsaturations, or 1 to about 5 unsaturations or 1 to about 3 unsaturations or 1 to 2 unsaturations, or alkyne having from 2 to about 20 carbon atoms and having from 1 to about 10 unsaturations, or 1 to about 5 unsaturations or 1 to about 3 unsaturations or 1 to 2 unsaturations, or substituted alkyne having 2 to about 20 carbon atoms and having from 1 to about 10 unsaturations, or 1 to about 5 unsaturations or 1 to about 3 unsaturations or 1 to 2 unsaturations, or a metabolic precursor thereof.

Another embodiment of the present invention is a physiologically acceptable ester of a compound of the formula:

A″-C(X²)(D¹)-CO₂H

wherein X² is alkyl of 1 to about 10 carbon atoms and D¹ is alkyl of 1 to about 10 carbon atoms and wherein A″ is alkyl of about 6 to about 20 carbon atoms, substituted alkyl of 6 to about 20 carbon atoms, alkene of about 6 to about 20 carbon atoms and having from 1 to about 10 unsaturations, or 1 to about 5 unsaturations or 1 to about 3 unsaturations or 1 to 2 unsaturations, or substituted alkene having from about 6 to about 20 carbon atoms and having from 1 to about 10 unsaturations, or 1 to about 5 unsaturations or 1 to about 3 unsaturations or 1 to 2 unsaturations, or alkyne having from 6 to about 20 carbon atoms and having from 1 to about 10 unsaturations, or 1 to about 5 unsaturations or 1 to about 3 unsaturations or 1 to 2 unsaturations, or substituted alkyne having 6 to about 20 carbon atoms and having from 1 to about 10 unsaturations, or 1 to about 5 unsaturations or 1 to about 3 unsaturations or 1 to 2 unsaturations, or a metabolic precursor thereof.

In a particular embodiment of the fatty acid compounds of the above formulas, A″ is selected from the group consisting of CₙH₂ₙ₊₁, CₙH₂ₙ₋₁, CₙH₂ₙ₋₃, and CₙH₂ₙ₋₅ wherein n is 1 to about 20 and wherein 1 to about 5 carbon atoms are optionally substituted with alkyl, branched alkyl, alkenyl, or alkynyl groups and the like.

Another embodiment of the present invention is a physiologically acceptable ester of a compound of the formula:

A″-(metabolic blocker)-CO₂H

wherein the metabolic blocker renders the energy requirement for beta-oxidation of the compound great enough to render the compound metabolically low caloric and wherein A″ is alkyl of 1 to about 20 carbon atoms, substituted alkyl of 1 to about 20 carbon atoms, alkene of 2 to about 20 carbon atoms and having from 1 to about 10 unsaturations, or 1 to about 5 unsaturations or 1 to about 3 unsaturations or 1 to 2 unsaturations, or substituted alkene having from 2 to about 20 carbon atoms and having from 1 to about 10 unsaturations, or 1 to about 5 unsaturations or 1 to about 3 unsaturations or 1 to 2 unsaturations, or alkyne having from 2 to about 20 carbon atoms and having from 1 to about 10 unsaturations, or 1 to about 5 unsaturations or 1 to about 3 unsaturations or 1 to 2 unsaturations, or substituted alkyne having 2 to about 20 carbon atoms and having from 1 to about 10 unsaturations, or 1 to about 5 unsaturations or 1 to about 3 unsaturations or 1 to 2 unsaturations, and wherein the metabolic blocker has the formula:

—(CH₂(CH₂)ₐCH(Y)(CH₂(CH₂)_{b}CH(Y))ₘ(CH₂(CH₂)_{c}CH(V)—

wherein m is 0 to 3 and wherein V and Y are independently alkyl having from 1 to about 10 carbon atoms and

(i) wherein, when the carbon atom comprising the V group is predominantly an S-enantiomer or is an S-enantiomer substantially free from R-enantiomer, the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and a, b and c are independently an integer of 1 to 5 or

(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer, the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and a, b and c are independently an integer of 1 to 5 and the b or c before the carbon atom that is the S-enantiomer is an integer of 1 or 3 or

(iii) wherein, when the carbon atom comprising the V group is predominantly an R-enantiomer or is an R-enantiomer substantially free from S-enantiomer and the carbon atoms comprising the Y-groups are predominantly an R-enantiomers or are R-enantiomers substantially free from S-enantiomers, at least one of a, b or c is an integer of 1 or 3 and the others are an integer of 1 to 5 and

wherein the carbon atoms not comprising the V group or the Y groups may be substituted with one or more substituents.

In a particular embodiment of the above the metabolic blocker has the formula:

—CH₂CH₂CH(Y)(CH₂CH₂CH(Y))ₘCH₂CH₂CH₂CH(Y)—.

In another particular embodiment of the above the metabolic blocker has the formula:

—CH₂CH₂CH(Y)(CH₂CH₂CH₂CH₂CH(Y))ₘCH₂CH₂CH₂CH(Y)—.

In a particular embodiment of the fatty acid compounds of the above formulas, A″ is selected from the group consisting of CₙH₂ₙ₊₁, CₙH₂ₙ₋₁, CₙH₂ₙ₋₃, and CₙH₂ₙ₋₅ wherein n is 1 to about 10 and wherein 1 to about 5 carbon atoms are optionally substituted with alkyl, branched alkyl, alkenyl, or alkynyl groups and the like.

Another embodiment of the present invention is a compound that is a fatty acid whose yield energy from beta-oxidation is sufficiently low so that the compound is rendered metabolically low caloric or non-caloric, or a physiologically acceptable ester thereof or a metabolic precursor thereof.

Another embodiment of the present invention is a compound of the formula:

A-(metabolic blocker)-CO₂H

wherein the metabolic blocker renders the yield energy from beta-oxidation of the compound sufficiently low that the compound is rendered metabolically low caloric or non-caloric and wherein the metabolic blocker has the formula:

—CH₂CH₂CH(Y)(CH₂(CH₂)_{z}CH(Y))ₘCH₂CH₂CH₂CH(V)—

wherein m is 0 to 3 and z is 1, 3, 5, or 7 and so forth and wherein V and Y are independently alkyl having from 1 to about 10 carbon atoms and

(i) wherein the carbon atom comprising the V group is predominantly an S-enantiomer or is an S-enantiomer substantially free from R-enantiomer and wherein the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and wherein the carbon atoms not comprising the V group or the Y groups may be substituted with one or more substituents or

(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and wherein the carbon atoms not comprising the V and the Y groups may be substituted with one or more substituents and wherein A″ is alkyl of 1 to about 20 carbon atoms, substituted alkyl of 1 to about 20 carbon atoms, alkene of 2 to about 20 carbon atoms and having from 1 to about 10 unsaturations, or 1 to about 5 unsaturations or 1 to about 3 unsaturations or 1 to 2 unsaturations, or substituted alkene having from 2 to about 20 carbon atoms and having from 1 to about 10 unsaturations, or 1 to about 5 unsaturations or 1 to about 3 unsaturations or 1 to 2 unsaturations, or alkyne having from 2 to about 20 carbon atoms and having from 1 to about 10 unsaturations, or 1 to about 5 unsaturations or 1 to about 3 unsaturations or 1 to 2 unsaturations, or substituted alkyne having 2 to about 20 carbon atoms and having from 1 to about 10 unsaturations, or 1 to about 5 unsaturations or 1 to about 3 unsaturations or 1 to 2 unsaturations, or a physiologically acceptable ester thereof or a metabolic precursor thereof.

In a particular embodiment of the above compound the metabolic blocker has the formula:

—CH₂CH₂CH(Y)(CH₂CH₂CH(Y))ₘCH₂CH₂CH₂CH(Y)—.

In another particular embodiment of the above compound the metabolic blocker has the formula:

—CH₂CH₂CH(Y)(CH₂CH₂CH₂CH₂CH(Y))ₘCH₂CH₂CH₂CH (Y)—.

In a particular embodiment of the above compounds, A is selected from the group consisting of CₙH₂ₙ₊₁, CₙH₂ₙ₋₁, CₙH₂ₙ₋₃, and CₙH₂ₙ₋₅ wherein n is 1 to about 15 and wherein 1 to about 5 carbon atoms are optionally substituted with, alkyl, branched alkyl, alkenyl, or alkynyl groups and the like.

Another embodiment of the present invention is a compound, which is a fatty acid substantially incapable of beta-oxidation, or a physiologically acceptable ester thereof or a metabolic precursor thereof.

Another embodiment of the present invention is a compound of the formula:

A′-(metabolic blocker)-CO₂H

wherein the metabolic blocker renders the compound substantially incapable of beta-oxidation and wherein A′ is alkyl of 1 to about 30 carbon atoms, substituted alkyl of 1 to about 30 carbon atoms, alkene of 2 to about 30 carbon atoms and having from 1 to about 10 unsaturations, or 1 to about 5 unsaturations or 1 to about 3 unsaturations or 1 to 2 unsaturations, or substituted alkene having from 2 to about 30 carbon atoms and having from 1 to about 10 unsaturations, or 1 to about 5 unsaturations or 1 to about 3 unsaturations or 1 to 2 unsaturations, or alkyne having from 2 to about 30 carbon atoms and having from 1 to about 10 unsaturations, or 1 to about 5 unsaturations or 1 to about 3 unsaturations or 1 to 2 unsaturations, or substituted alkyne having 2 to about 30 carbon atoms and having from 1 to about 10 unsaturations, or 1 to about 5 unsaturations or 1 to about 3 unsaturations or 1 to 2 unsaturations, or a physiologically acceptable ester thereof. In a particular embodiment, A′ is selected from the group consisting of CₙH₂ₙ₊₁, CₙH₂ₙ₋₁, CₙH₂ₙ₋₃, and CₙH₂ₙ₋₅ wherein n is about 8 to about 20 and wherein 1 to about 5 carbon atoms are, alkyl, branched alkyl, alkenyl, or alkynyl and the like.

In one embodiment of the above compound, the metabolic blocker has the formula:

—C(X¹)(D)-,

wherein X¹ is alkyl of 1 to about 10 carbon atoms and D is hydrogen or alkyl of 1 to about 10 carbon atoms and wherein, when D is hydrogen, the metabolic blocker is an enantiomeric mixture that comprises at least 50% of the S-enantiomer up to an S-enantiomer substantially free from R-enantiomer and wherein A′ is alkyl of about 4 to about 20 carbon atoms, substituted alkyl of about 4 to about 20 carbon atoms, alkene of about 4 to about 20 carbon atoms and having from 1 to about 10 unsaturations, or 1 to about 5 unsaturations or 1 to about 3 unsaturations or 1 to 2 unsaturations, or substituted alkene having from about 4 to about 20 carbon atoms and having from 1 to about 10 unsaturations, or 1 to about 5 unsaturations or 1 to about 3 unsaturations or 1 to 2 unsaturations, or alkyne having from 4 to about 20 carbon atoms and having from 1 to about 10 unsaturations, or 1 to about 5 unsaturations or 1 to about 3 unsaturations or 1 to 2 unsaturations, or substituted alkyne having 4 to about 20 carbon atoms and having from 1 to about 10 unsaturations, or 1 to about 5 unsaturations or 1 to about 3 unsaturations or 1 to 2 unsaturations, or a physiologically acceptable ester thereof or a metabolic precursor thereof.

Another embodiment of the present invention is a compound, which is a fatty acid having a yield energy from beta-oxidation that is low enough to render the fatty acid metabolically low caloric, or a physiologically acceptable ester thereof or a metabolic precursor thereof.

Another embodiment of the present invention is a compound of the formula:

A″-(metabolic blocker)-CO₂H

wherein the metabolic blocker renders the energy requirement for beta-oxidation of the compound great enough to render the compound metabolically low caloric and wherein A″ is alkyl of 1 to about 20 carbon atoms, substituted alkyl of 1 to about 20 carbon atoms, alkene of 2 to about 20 carbon atoms and having from 1 to about 10 unsaturations, or 1 to about 5 unsaturations or 1 to about 3 unsaturations or 1 to 2 unsaturations, or substituted alkene having from 2 to about 20 carbon atoms and having from 1 to about 10 unsaturations, or 1 to about 5 unsaturations or 1 to about 3 unsaturations or 1 to 2 unsaturations, or alkyne having from 2 to about 20 carbon atoms and having from 1 to about 10 unsaturations, or 1 to about 5 unsaturations or 1 to about 3 unsaturations or 1 to 2 unsaturations, or substituted alkyne having 2 to about 20 carbon atoms and having from 1 to about 10 unsaturations, or 1 to about 5 unsaturations or 1 to about 3 unsaturations or 1 to 2 unsaturations, or a physiologically acceptable ester thereof. In a particular embodiment of the above compound, A″ is selected from the group consisting of CₙH₂ₙ₊₁, CₙH₂ₙ₋₁, CₙH₂ₙ₋₃, and CₙH₂ₙ₋₅ wherein n is 1 to about 20 and wherein 1 to about 5 carbon atoms are optionally substituted with, alkyl, branched alkyl, alkenyl, or alkynyl and the like.

In one embodiment of the above, the metabolic blocker has the formula:

—CH₂CH₂CH(Y)(CH₂(CH₂)_{z}CH(Y))ₘCH₂CH₂CH₂CH(V)—

wherein m is 0 to 3 and z is 1, 3, 5, 7 and so forth and wherein V and Y are independently alkyl having from 1 to about 10 carbon atoms and

(i) wherein the carbon atom comprising the V group is predominantly an S-enantiomer or is an S-enantiomer substantially free from R-enantiomer and wherein the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and wherein the carbon atoms not comprising the V group or the Y groups may be substituted with one or more substituents or

(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and wherein the carbon atoms not comprising the V and the Y groups may be substituted with one or more substituents.

In a particular embodiment of the above compound the metabolic blocker has the formula:

—CH₂CH₂CH(Y)(CH₂CH₂CH(Y))ₘCH₂CH₂CH₂CH(Y)—.

In another particular embodiment of the above compound the metabolic blocker has the formula:

—CH₂CH₂CH(Y)(CH₂CH₂CH₂CH₂CH(Y))ₘCH₂CH₂CH₂CH(Y)—.

Another embodiment of the present invention is a method for rendering an organic acid compound metabolically low caloric or non-caloric. The method comprises introducing into the compound one or more substituents at a carbon atom that is alpha to a carboxyl group of the organic acid compound. The substituent renders the yield energy from beta-oxidation of the organic acid compound sufficiently low that the compound is rendered metabolically low caloric or non-caloric.

Another embodiment of the present invention involves polyhydric alcohol esters of the compounds mentioned above.

Another embodiment of the present invention is a food composition comprising an edible fat and a compound as described above.

Another embodiment of the present invention is a food composition comprising a non-fat ingredient and a fat ingredient at least a portion of which is a compound as described above.

Fatty Acid Compounds of the Invention

The fatty acid compounds are organic monobasic acids that comprise a carboxylic acid moiety and a hydrocarbon moiety having at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 carbon atoms and may be in the range of about 6 to about 30 carbon atoms or more, about 10 to about 30 carbon atoms or more, about 15 to about 30 carbon atoms or more. The compounds may be branched or unbranched, saturated (0 unsaturations) or unsaturated. The unsaturated fatty acid compounds may comprise from 1 to about 10, 1 to about 5, 1 to about 3 unsaturations, 1 to 2 unsaturations, which may be double bonds, triple bonds or a combination of both. Generally, the hydrocarbon moiety is free from cyclic moieties or rings. Carbon atoms of the hydrocarbon moiety may comprise one or more substituents such as, for example, alkyl, alkenyl, alkynyl and the like. In some embodiments, the number of substituents present in the fatty acid compounds other than alkyl is 1 to about 10, or 1 to about 5.

“Alkyl” means a branched or unbranched saturated monovalent hydrocarbon radical containing 1 to 30 or more carbon atoms, such as methyl, ethyl, propyl, tert-butyl, n-hexyl, iso-hexyl, n-octyl, iso-octyl, and so forth. Alkyl includes lower alkyl. “Lower alkyl” means a branched or unbranched saturated monovalent hydrocarbon radical containing 1 to 10 carbon atoms, such as methyl, ethyl, propyl, isopropyl, tert-butyl, iso-butyl, n-pentyl, iso-pentyl, and so forth. In some embodiments, the alkyl substituent is lower alkyl, such as, e.g., methyl.

“Alkene” means a branched or unbranched unsaturated hydrocarbon radical containing at least one double or ethenylic bond and 2 to 30 or more carbon atoms and includes lower alkene, unless otherwise indicated. “Lower alkene” means a branched or unbranched unsaturated hydrocarbon radical containing at least one double or ethenylic bond and 2 to 6 carbon atoms, unless otherwise indicated. One or more carbon atoms of the hydrocarbon are optionally substituted.

“Alkyne” means a branched or unbranched unsaturated hydrocarbon radical containing at least one triple or ethynylic bond and 2 to 30 or more carbon atoms and includes lower alkyne, unless otherwise indicated. “Lower alkyne” means a branched or unbranchedunsaturated hydrocarbon radical containing at least one triple or ethynylic bond and 2 to 6 carbon atoms, unless otherwise indicated. One or more carbon atoms of the hydrocarbon are optionally substituted.

“Substituted” means that one or more carbon atoms of the alkyl, alkene or alkyne may comprise one or more substituents such as, for example, alkyl, alkenyl, alkynyl and the like. “Optional” or “optionally” means that the subsequently described event or circumstance may or may not occur, and that the description includes instances where said event or circumstance occurs and instances in which it does not. For example, “optionally substituted” means that a substituent may or may not be present on the carbon atom of the hydrocarbon. The fatty acid compounds of the invention include physiologically acceptable esters thereof or a metabolic precursor thereof.

“Physiologically acceptable” means that the ester may be consumed without deleterious effects on the consumer. Such deleterious effects include toxicity, palatability, decrease in the availability of the fat-soluble vitamins A, D, E, and K, diarrhea, loose stools, gas and abdominal cramping and the like. In some embodiments, the esters are esters of a polyhydric alcohol, which is any aliphatic or aromatic compound containing at least two free hydroxyl groups. In some embodiments the polyhydric alcohol for ester formation is glycerol, which yields triglycerides from the fatty acid compounds of the invention. The polyhydric alcohol esters of the present fatty acid compounds may be mono-, di- and tri-esters. In some embodiments the polyhydric alcohol esters are tri-esters.

“Metabolic precursor” means that the compound in question, when ingested, results in a compound of the invention as a fat substitute by means of metabolic processes such as, for example, oxidation processes. For example, R,R,S-phytol is a metabolic precursor to R,R,S-phytanic acid and subsequently R,R,S-pristanic acid as a result of metabolic oxidation and other processes. Furthermore, R,R,R-phytol is a metabolic precursor to R,R,R-phytanic acid and subsequently R,R,R-pristanic acid as a result of such processes.

Generally speaking, a fat substitute providing fewer calories than a conventional triglyceride should not be directly absorbed through the intestinal wall. While some types of fat substitutes may be non-digestible, they are not of sufficiently high molecular weight to prevent them from being absorbed through the intestinal wall. The threshold molecular weight of non-absorbability for lipophilic molecules appears to be about 600. Furthermore, it is often desirable that the fat substitute have the properties of a triglyceride oil when formulated into food compositions. A fat substitute having these characteristics would likely enjoy greater consumer acceptance than many of the currently known mimetics. Triglyceride esters of the present fatty acid compounds are particularly attractive because they have a number of properties of known fatty acid triglycerides but, after hydrolysis of such triglyceride during metabolic processing in vivo, the resulting free fatty acids of the invention have the low caloric or non-caloric benefits described above.

As mentioned above, one embodiment of the present invention is a method of reducing or eliminating metabolic caloric content of a food composition comprising an edible fat. The method comprises substituting, for at least a portion of the edible fat of the food composition, a compound that is a fatty acid whose yield energy from beta-oxidation is sufficiently low that the compound is rendered metabolically low caloric or non-caloric, or a physiologically acceptable ester thereof or a metabolic precursor thereof.

A specific embodiment of the present invention involves a compound of the formula:

A-(metabolic blocker)-CO₂H

wherein the metabolic blocker renders the yield energy from beta-oxidation of the compound sufficiently low that the compound is rendered metabolically low caloric or non-caloric and wherein A is

(i) alkyl of 1 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 carbon atoms and may be in the range of about 6 to about 30 carbon atoms or more, about 10 to about 30 carbon atoms or more, about 15 to about 30 carbon atoms or more;

(ii) substituted alkyl of 1 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 carbon atoms and may be in the range of about 6 to about 30 carbon atoms or more, about 10 to about 30 carbon atoms or more, about 15 to about 30 carbon atoms or more;

(iii) alkene of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 carbon atoms and may be in the range of about 6 to about 30 carbon atoms or more, about 10 to about 30 carbon atoms or more, about 15 to about 30 carbon atoms or more and having from 1 to about 10 unsaturations, 1 to about 5 unsaturations, 1 to about 3 unsaturations, 1 to 2 unsaturations, or

(iv) substituted alkene having from 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 carbon atoms and may be in the range of about 6 to about 30 carbon atoms or more, about 10 to about 30 carbon atoms or more, about 15 to about 30 carbon atoms or more and having from 1 to about 10 unsaturations, 1 to about 5 unsaturations, 1 to about 3 unsaturations, 1 to 2 unsaturations;

(v) alkyne of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations, 1 to about 5 unsaturations, 1 to about 3 unsaturations, 1 to 2 unsaturations, or

(vi) substituted alkyne having from 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations, 1 to about 5 unsaturations, 1 to about 3 unsaturations, 1 to 2 unsaturations,

or a physiologically acceptable ester thereof or a metabolic precursor thereof. In general, the number of carbon atoms in A is dependent on the number of carbon atoms in the metabolic blocker since the overall number of carbon atoms of the hydrocarbon portion of the fatty acid compound is as set forth above with reference to the discussion regarding fatty acid compounds. Furthermore, the number of unsaturations in A is also dependent on the number of carbon atoms of the hydrocarbon portion of the fatty acid compound.

The term “metabolically non-caloric” means that a compound that is oxidized yields less calories than the amount of calories that are used in the process of oxidation.

The term “metabolically low caloric” means that a compound as described herein will yield less calories than a corresponding “natural” fatty acid as normally found in the diet.

In one embodiment of the above compound the metabolic blocker has the formula: —C(X)(D)-, wherein X is alkyl of 1 to about 10 carbon atoms and D is hydrogen or alkyl of 1 to about 10 carbon atoms and wherein, when D is hydrogen, the metabolic blocker comprises predominantly an S-enantiomer, that is, an enantiomeric mixture that comprises at least 50% of the S-enantiomer, up to an S-enantiomer substantially free from R-enantiomer, and wherein A is

(i) alkyl of 5 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 25 carbon atoms or more, about 10 to about 20 carbon atoms or more, about 15 to about 20 carbon atoms or more;

(ii) substituted alkyl of 5 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 25 carbon atoms or more, about 10 to about 20 carbon atoms or more, about 15 to about 20 carbon atoms or more;

(iii) alkene of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 25 carbon atoms or more, about 10 to about 20 carbon atoms or more, about 15 to about 20 carbon atoms or more and having from 1 to about 10 unsaturations, from 1 to about 10 unsaturations, or 1 to about 5 unsaturations or 1 to about 3 unsaturations or 1 to 2 unsaturations, or

(iv) substituted alkene having from 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 25 carbon atoms or more, about 10 to about 20 carbon atoms or more, about 15 to about 20 carbon atoms or more and having from 1 to about 10 unsaturations, from 1 to about 10 unsaturations, or 1 to about 5 unsaturations or 1 to about 3 unsaturations or 1 to 2 unsaturations. In some embodiments, X is alkyl of 1 to about 5 carbon atoms, for example, X is methyl. In some embodiments, when D is alkyl, D is alkyl of 1 to about 5 carbon atoms, for example, D is methyl.

The term “enantiomer” as used herein refers to the stereochemical or stereoisomeric arrangement of substituents on a particular carbon atom of a molecule. Such a carbon atom is also referred to herein as a chiral center.

The phrase “predominantly an S-enantiomer” means an enantiomeric mixture that comprises at least 50% of an S-enantiomer, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, of an S-enantiomer.

The phrase “predominantly an R-enantiomer” means an enantiomeric mixture that comprises at least 50% of an R-enantiomer, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, of an R-enantiomer.

“Substantially free from R-enantiomer” means that the amount of R-isomer of the particular carbon atom or chiral center of the fatty acid compound in question, if any, present is insufficient to render the metabolic caloric value of the fatty acid compound greater than low caloric or non-caloric. In some embodiments, the fatty acid compound contains no more than about 1% of the corresponding R-isomer at the particular carbon atom.

“Substantially free from S-enantiomer” means that the amount of S-isomer of the particular carbon atom or chiral center of the fatty acid compound in question, if any, present is insufficient by itself to render the metabolic caloric value of the fatty acid compound low caloric or non-caloric. In some embodiments, the fatty acid compound contains no more than about 1% of the corresponding S-isomer at the particular carbon atom.

In a particular embodiment of the fatty acid compounds of the above formula, A is selected from the group consisting of CₙH₂ₙ₊₁, CₙH₂ₙ₋₁, CₙH₂ₙ₋₃, and CₙH₂ₙ₋₅ wherein n is an integer from about 5 to about 30, 6 to about 25, 10 to about 20 and wherein 1 to 5 carbon atoms are optionally substituted with one or more substituents.

Another specific embodiment of the present invention involves a compound of the formula:

A″-(metabolic blocker)-CO₂H

wherein the metabolic blocker renders the energy requirement for beta-oxidation of the compound great enough to render the compound metabolically low caloric and wherein the metabolic blocker has the formula:

—(CH₂(CH₂)ₐCH(Y)(CH₂(CH₂)_{b}CH(Y))ₘ(CH₂(CH₂)_{c}CH (V)—

wherein m is 0 to 3 and wherein V and Y are independently alkyl having from 1 to about 10 carbon atoms and

(i) wherein when the carbon atom comprising the V group is predominantly an S-enantiomer or is an S-enantiomer substantially free from R-enantiomer, the carbon atoms comprising the Y groups are independently predominantly R-enantiomers or R-enantiomers substantially free from S-enantiomers or predominantly S-enantiomers or are S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and a, b and c are independently 1, 2, 3, 4 or 5 or

(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and a, b and c are independently an 1, 2, 3, 4 or 5 and the b or c before the carbon atom that is the S-enantiomer is an integer of 1 or 3 or

(iii) wherein, when the carbon atom comprising the V group is predominantly an R-enantiomer or is an R-enantiomer substantially free from S-enantiomer and the carbon atoms comprising the Y-groups are predominantly an R-enantiomer or are R-enantiomers substantially free from S-enantiomers, at least one of a, b or c is 1 or 3 and the remaining a, b and/or c are independently 1, 2, 3, 4 or 5 and wherein the carbon atoms not comprising the V group or the Y groups may be substituted with one or more substituents and

wherein A″ is

(i) alkyl of 1 to about 20 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14 carbon atoms, at least about 15 carbon atoms, at least about 16 carbon atoms, at least about 17 carbon atoms, at least about 18 carbon atoms, at least about 19 carbon atoms, and may be in the range of about 1 to about 15 carbon atoms or more, about 2 to about 8 carbon atoms or more, about 3 to about 7 carbon atoms or more;

(ii) substituted alkyl of 1 to about 15 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14 carbon atoms, at least about 15 carbon atoms, at least about 16 carbon atoms, at least about 17 carbon atoms, at least about 18 carbon atoms, at least about 19 carbon atoms, and may be in the range of about 1 to about 115 carbon atoms or more, about 2 to about 8 carbon atoms or more, about 3 to about 7 carbon atoms or more;

(iii) alkene of 2 to about 15 carbon atoms, in some embodiments, at least about 2, at least about 3, at least about 4, at least about 5, at least about 6, at least about 7, at least about 8, at least about 9, at least about 10, at least about 11, at least about 12, at least about 13, at least about 14 carbon atoms, at least about 15 carbon atoms, at least about 16 carbon atoms, at least about 17 carbon atoms, at least about 18 carbon atoms, at least about 19 carbon atoms, and may be in the range of about 2 to about 15 carbon atoms or more, about 3 to about 8 carbon atoms or more, about 4 to about 7 carbon atoms or more and having from 1 to about 10 unsaturations, from 1 to about 5 unsaturations or

(iv) substituted alkene having from 2 to about 15 carbon atoms, in some embodiments, at least about 2, at least about 3, at least about 4, at least about 5, at least about 6, at least about 7, at least about 8, at least about 9, at least about 10, at least about 11, at least about 12, at least about 13, at least about 14 carbon atoms, at least about 15 carbon atoms, at least about 16 carbon atoms, at least about 17 carbon atoms, at least about 18 carbon atoms, at least about 19 carbon atoms, and may be in the range of about 2 to about 15 carbon atoms or more, about 3 to about 8 carbon atoms or more, about 4 to about 7 carbon atoms or more and having from 1 to about 10 unsaturations, from 1 to about 5 unsaturations, from 1 to about 3 unsaturations. In some embodiments, A″ is alkyl of 1 to about 5 carbon atoms, or 1 to 3 carbon atoms; substituted alkyl of 1 to about 10 carbon atoms, or 1 to 3 carbon atoms; alkene of 2 to about 10 carbon atoms and having from 1 to about 5 unsaturations, or 1 to 3 unsaturations or 1 to 2 unsaturations, or 2 to 6 carbon atoms and having from 1 to about 3 unsaturations or 1 to 2 unsaturations; or substituted alkene having from 2 to about 10 carbon atoms and having from 1 to about 5 unsaturations, or 1 to about 3 unsaturations, or 1 to 2 unsaturations, or 2 to 6 carbon atoms and having from 1 to about 3 unsaturations or 1 to 2 unsaturations.

In another specific embodiment of the fatty acid compounds of the invention, the metabolic blocker has the formula:

—CH₂CH₂CH(Y)(CH₂(CH₂)ₙCH(Y))ₘCH₂CH₂CH₂CH(V)—

wherein m is 0 to 3, n is 1 or 3 and V and Y are independently alkyl having from 1 to about 10 carbon atoms and

(i) wherein the carbon atom with the V group as a substituent is predominantly an S-enantiomer or is an S-enantiomer substantially free of R-enantiomer and the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) or

(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and wherein the carbon atoms not comprising the Y groups may be substituted with one or more substituents and

wherein A″ is

(i) alkyl of 1 to about 20 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more;

(ii) substituted alkyl of 1 to about 20 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more;

(iii) alkene of 2 to about 20 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more and having from 1 to about 10 unsaturations, 1 to about 5 unsaturations, 1 to about 3 unsaturations, 1 to 2 unsaturations, or

(iv) substituted alkene having from 2 to about 20 carbon atoms, in some embodiments, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more and having from 1 to about 10 unsaturations, from 1 to about 5 unsaturations, from 1 to about 3 unsaturations, from 1 to 2 unsaturations. In some embodiments, A″ is alkyl of 1 to about 5 carbon atoms, or 1 to 3 carbon atoms; substituted alkyl of 1 to about 10 carbon atoms, or 1 to 3 carbon atoms; alkene of 2 to about 10 carbon atoms and having from 1 to about 5 unsaturations or 1 to about 3 unsaturations or 1 to 2 unsaturations, or 2 to 6 carbon atoms and having from 1 to about 3 unsaturations or 1 to 2 unsaturations; or substituted alkene having from 2 to about 10 carbon atoms and having from 1 to about 5 unsaturations or 1 to about 3 unsaturations or 1 to 2 unsaturations, or 2 to 6 carbon atoms and having from 1 to about 3 unsaturations or 1 to 2 unsaturations. In some embodiments, Y is alkyl of 1 to about 5 carbon atoms; for example, Y is methyl.

When m is 0, the metabolic blocker has the formula:

—CH₂CH₂CH(Y)CH₂CH₂CH₂CH(V)—.

When m is 1 and n is 1, the metabolic blocker has the formula:

—CH₂CH₂CH(Y)CH₂CH₂CH(Y)CH₂CH₂CH₂CH(V)—.

When m is 2 and n is 1, the metabolic blocker has the formula:

—CH₂CH₂CH(Y)(CH₂CH₂CH(Y)(CH₂CH₂CH(Y)CH₂CH₂CH₂CH(V)—.

When m is 3 and n is 1, the metabolic blocker has the formula:

—CH₂CH₂CH(Y)CH₂CH₂CH(Y)CH₂CH₂CH(Y)CH₂CH₂CH(Y)CH₂CH₂CH₂CH(V)—.

When m is 1 and n is 3, the metabolic blocker has the formula:

—CH₂CH₂CH(Y)CH₂CH₂CH₂CH₂CH(Y)CH₂CH₂CH₂CH(V)—.

When m is 2 and n is 3, the metabolic blocker has the formula:

—CH₂CH₂CH(Y)CH₂CH₂CH₂CH₂CH(Y)CH₂CH₂CH₂CH₂CH(Y)CH₂CH₂CH₂CH(V)—.

When m is 3 and n is 3, the metabolic blocker has the formula:

—CH₂CH₂CH(Y)CH₂CH₂CH₂CH₂CH(Y)CH₂CH₂CH₂CH₂CH(Y)CH₂CH₂CH₂CH₂CH(Y)CH₂CH₂CH₂CH(V)—.

In some embodiments, A″ is alkyl of 1 to about 5 carbon atoms, or 1 to 3 carbon atoms; substituted alkyl of 1 to about 10 carbon atoms, or 1 to 3 carbon atoms; alkene of 2 to about 10 carbon atoms and having from 1 to about 5 unsaturations or 1 to about 3 unsaturations or 1 to 2 unsaturations, or 2 to 6 carbon atoms and having from 1 to about 3 unsaturations or 1 to 2 unsaturations; or substituted alkene having from 2 to about 10 carbon atoms and having from 1 to about 5 unsaturations or 1 to about 3 unsaturations or 1 to 2 unsaturations, or 2 to 6 carbon atoms and having from 1 to about 3 unsaturations or 1 to 2 unsaturations.

In a particular embodiment of the fatty acid compounds of the above formulas, A″ is selected from the group consisting of CₙH₂ₙ₊₁, CₙH₂ₙ₋₁, C₁H₂ₙ₋₃, and CₙH₂ₙ₋₅ wherein n is 1 to 20, or 1 to 10, and wherein 1 to 5, or 1 to 3, carbon atoms are optionally substituted with, alkyl, alkenyl, alkynyl and the like.

The A (or variants thereof such as, e.g., A′ and A″ and so forth) moiety may be a “short chain alkyl (alkenyl, alkynyl)” or a “long chain alkyl (alkenyl, alkynyl)” where alkenyl and alkynyl have definitions corresponding to the alkyl counterparts. “Short chain alkyl” means an alkyl containing from about 4 to about 10 carbon atoms. “Long chain alkyl” means an alkyl containing more than 10 carbon atoms. Examples of A moieties for the fatty acid compounds of the aforementioned formulas, by way of illustration and not limitation, include (numbering begins with the carbon atom that is attached to the metabolic blocker), n-butyl, n-hexyl, n-octyl, n-decyl, n-dodecyl, n-tetradecyl, n-pentadecyl, n-hexadecyl, n-heptadecyl, n-octadecyl, n-nonadecyl, n-tetradecenyl, n-hexadecenyl, 7-hexadecenyl, 11-hexadecenyl, 5-tetradecynyl, 10-hydroxy-7-hexadecyl, 7,10-hexadecadienyl, 7,10,13-hexadecatrienyl, hexadeca-9,11,13-trienyl, 10-octadecenyl, and the like.

In some embodiments, it is desirable to have a fatty acid for which w-oxidation is avoided or the possibility thereof is reduced. w-Oxidation occurs when a- or b-oxidation cannot occur, for example, when the a-carbon is in the S configuration with a free carboxylic acid. To address this issue in these embodiments, in one approach the chiral carbon atom at the w-end of the molecule is designed to have a stereochemical configuration that is opposite to that of the other chiral centers. For example, if all of the other chiral centers are in the S-configuration, the w chiral center is in the R-configuration. In an alternate approach, a quaternary center on both ends of the fatty acid may be employed to avoid w-oxidation. If the ω chiral carbon is the same configuration as the other chiral carbons, e.g., S, then, the resulting product has R chiral centers when oxidation occurs from the ω-end, thereby using more ATP.

Specific Embodiments of Compounds in Accordance with the Present Invention

One series of compounds in accordance with the present invention includes compounds and their physiologically acceptable esters, or a metabolic precursor of such compounds, represented by the formula:

W—C(CH₃)(CH₃)—CO₂H

wherein W is alkyl of 1 to about 30 carbon atoms, substituted alkyl of 1 to about 30 carbon atoms, alkene of 2 to about 30 carbon atoms and having from 1 to about 10 unsaturations or 1 to about 5 unsaturations or 1 to about 3 unsaturations or 1 to 2 unsaturations, or substituted alkene having from 2 to about 30 carbon atoms and having from 1 to about 10 unsaturations or 1 to about 5 unsaturations or 1 to about 3 unsaturations or 1 to 2 unsaturations, alkyne of 2 to about 30 carbon atoms and having from 1 to about 10 unsaturations or 1 to about 5 unsaturations or 1 to about 3 unsaturations or 1 to 2 unsaturations, or substituted alkyne having from 2 to about 30 carbon atoms and having from 1 to about 10 unsaturations or 1 to about 5 unsaturations or 1 to about 3 unsaturations or 1 to 2 unsaturations, or a physiologically acceptable ester thereof. In some embodiments, W is (i) alkyl of 1 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more; (ii) substituted alkyl of 1 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more; (iii) alkene of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations, or (iv) substituted alkene having from 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations; (v) alkyne of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations, or (vi) substituted alkyne having from 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations.

Another series of compounds in accordance with the present invention includes compounds and their physiologically acceptable esters, or a metabolic precursor such compound, represented by the formula:

Z-CH(CH₃)—CO₂H

wherein the alpha carbon is predominantly an S-enantiomer or is an S-enantiomer substantially free from R-enantiomer and Z is alkyl of 1 to about 30 carbon atoms, substituted alkyl of 1 to about 30 carbon atoms, alkene of 2 to about 30 carbon atoms and having from 1 to about 10 unsaturations or 1 to about 5 unsaturations or 1 to about 3 unsaturations or 1 to 2 unsaturations, or substituted alkene having from 2 to about 30 carbon atoms and having from 1 to about 10 unsaturations or 1 to about 5 unsaturations or 1 to about 3 unsaturations or 1 to 2 unsaturations, alkyne of 2 to about 30 carbon atoms and having from 1 to about 10 unsaturations or 1 to about 5 unsaturations or 1 to about 3 unsaturations or 1 to 2 unsaturations, or substituted alkyne having from 2 to about 30 carbon atoms and having from 1 to about 10 unsaturations or 1 to about 5 unsaturations or 1 to about 3 unsaturations or 1 to 2 unsaturations, or a physiologically acceptable ester thereof. In some embodiments, Z is (i) alkyl of 1 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more; (ii) substituted alkyl of 1 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more; (iii) alkene of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations, or (iv) substituted alkene having from 2 to about 30 carbon atoms, in some embodiments, at least about 6, at least about 7, at least about 8, at least about 9, at least about 10, at least about 11, at least about 12, at least about 13, at least about 14, at least about 5, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations; (v) alkyne of 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations, or (vi) substituted alkyne having from 2 to about 30 carbon atoms, in some embodiments, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 30 carbon atoms or more, about 10 to about 30 carbon atoms or more, about 15 to about 30 carbon atoms or more and having from 1 to about 10 unsaturations or 1 to about 5 unsaturations or 1 to about 3 unsaturations or 1 to 2 unsaturations.

Another series of compounds in accordance with the present invention includes compounds and their physiologically acceptable esters, or a metabolic precursor thereof, represented by the formula:

Q-CH₂CH₂CH(CH₃)CH₂CH₂CH(CH₃)CH₂CH₂CH₂CH(CH₃)—CO₂H

(i) wherein the carbon atom alpha to the carboxyl group is predominantly an S-enantiomer or is an S-enantiomer substantially free of R-enantiomer and the other carbon atoms comprising the methyl groups are independently R-enantiomers substantially free from S-enantiomers or S-enantiomers substantially free from R-enantiomers or one or more of the carbon atoms comprising the methyl groups may be a combination of S-enantiomers and R-enantiomers (thereby rendering the fatty acid compound wholly or partially racemic with respect to such carbon atom(s)) or

(ii) wherein, when the alpha carbon atom comprising the methyl group is predominantly an R-enantiomer or is an R-enantiomer substantially free from S-enantiomer and one of the other carbon atoms comprising a methyl group is predominantly an S-enantiomer or is an S-enantiomer substantially free from R-enantiomer and the other carbon atoms comprising the methyl group are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and wherein Q is selected from the group consisting of alkyl of 1 to about 20 carbon atoms, substituted alkyl of 1 to about 20 carbon atoms, alkene of 2 to about 20 carbon atoms and having from 1 to about 5 unsaturations or 1 to about 3 unsaturations or 1 to 2 unsaturations, or substituted alkene having from 2 to about 20 carbon atoms and having from 1 to about 5 unsaturations or 1 to about 3 unsaturations or 1 to 2 unsaturations.

In some embodiments of the above, Q is (i) alkyl of 1 to about 20 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more; (ii) substituted alkyl of 1 to about 20 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more; (iii) alkene of 2 to about 20 carbon atoms, in some embodiments, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more and having from 1 to about 10 unsaturations or 1 to about 5 unsaturations or 1 to about 3 unsaturations or 1 to 2 unsaturations, or (iv) substituted alkene having from 2 to about 20 carbon atoms, in some embodiments, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, carbon atoms and may be in the range of about 6 to about 20 carbon atoms or more, about 10 to about 20 carbon atoms or more, about 15 to about 20 carbon atoms or more and having from 1 to about 10 unsaturations, from 1 to about 5 unsaturations or 1 to about 3 unsaturations or 1 to 2 unsaturations. In some embodiments, Q is alkyl of 1 to about 5 carbon atoms, 1 to 3 carbon atoms; substituted alkyl of 1 to about 10 carbon atoms, 1 to 3 carbon atoms; alkene of 2 to about 10 carbon atoms and having from 1 to about 5 unsaturations or 1 to about 3 unsaturations or 1 to 2 unsaturations, 2 to 6 carbon atoms and having from 1 to about 3 unsaturations or 1 to 2 unsaturations; or substituted alkene having from 2 to about 10 carbon atoms and having from 1 to about 5 unsaturations or 1 to about 3 unsaturations or 1 to 2 unsaturations, 2 to 6 carbon atoms and having from 1 to about 3 unsaturations or 1 to 2 unsaturations.

Another series of compounds in accordance with the present invention includes compounds and their physiologically acceptable esters, or a metabolic precursor thereof, represented by the formula:

Q′-CH₂CH₂CH₂CH₂CH(CH₃)CH₂CH₂CH₂CH₂CH(CH₃)CH₂CH₂CH₂CH(CH₃)—CO₂H

(i) wherein the carbon atom alpha to the carboxyl group is predominantly an S-enantiomer or is an S-enantiomer substantially free of R-enantiomer and the other carbon atoms comprising the methyl groups are independently R-enantiomers substantially free from S-enantiomers or S-enantiomers substantially free from R-enantiomers or one or more of the carbon atoms comprising the methyl groups may be a combination of S-enantiomers and R-enantiomers (thereby rendering the fatty acid compound wholly or partially racemic with respect to such carbon atom(s)) or

(ii) wherein, when the alpha carbon atom comprising the methyl group is predominantly an R-enantiomer or is an R-enantiomer substantially free from S-enantiomer and one of the other carbon atoms comprising a methyl group is predominantly an S-enantiomer or is an S-enantiomer substantially free from R-enantiomer and the other carbon atoms comprising the methyl groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and wherein Q′ is selected from the group consisting of alkyl of 1 to about 15 carbon atoms, substituted alkyl of 1 to about 15 carbon atoms, alkene of 2 to about 15 carbon atoms and having from 1 to about 5 unsaturations or 1 to about 3 unsaturations or 1 to 2 unsaturations, or substituted alkene having from 2 to about 15 carbon atoms and having from 1 to about 5 unsaturations or 1 to about 3 unsaturations or 1 to 2 unsaturations.

In some embodiments, Q′ is (i) alkyl of 1 to about 15 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14 carbon atoms and may be in the range of about 2 to about 14 carbon atoms or more, about 3 to about 12 carbon atoms or more, about 5 to about 10 carbon atoms or more; (ii) substituted alkyl of 1 to about 15 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14 carbon atoms and may be in the range of about 2 to about 14 carbon atoms or more, about 3 to about 12 carbon atoms or more, about 5 to about 10 carbon atoms or more; (iii) alkene of 2 to about 15 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14 carbon atoms and may be in the range of about 2 to about 14 carbon atoms or more, about 3 to about 12 carbon atoms or more, about 5 to about 10 carbon atoms or more and having from 1 to about 10 unsaturations, 1 to about 5 unsaturations or 1 to about 3 unsaturations or 1 to 2 unsaturations, or (iv) substituted alkene having from 2 to about 15 carbon atoms, in some embodiments, at least about 1, at least about 2, at least about 3, at least about 4, at least about 5, at least about 6, at least about 7, at least about 8, at least about 9, at least about 10, at least about 11, at least about 12, at least about 13, at least about 14 carbon atoms and may be in the range of about 2 to about 14 carbon atoms or more, about 3 to about 12 carbon atoms or more, about 5 to about 10 carbon atoms or more and having from 1 to about 10 unsaturations, from 1 to about 5 unsaturations or 1 to about 3 unsaturations or 1 to 2 unsaturations. In some embodiments, Q′ is alkyl of 1 to about 10 carbon atoms, 1 to 3 carbon atoms; substituted alkyl of 1 to about 10 carbon atoms, 1 to 3 carbon atoms; alkene of 2 to about 10 carbon atoms and having from 1 to about 5 unsaturations or 1 to about 3 unsaturations or 1 to 2 unsaturations, 2 to 6 carbon atoms and having from 1 to about 3 unsaturations or 1 to 2 unsaturations; or substituted alkene having from 2 to about 10 carbon atoms and having from 1 to about 5 unsaturations or 1 to about 3 unsaturations or 1 to 2 unsaturations, 2 to 6 carbon atoms and having from 1 to about 3 unsaturations or 1 to 2 unsaturations.

With respect to fatty acid compounds themselves that comprise multiple repeating methyl substituted carbon atoms in a chain, R,R,S-phytanic acid, R,R,R-phytanic acid, R,R,S-pristanic acid and R,R,R-pristanic acid are specifically excluded except for enantiomerically purified forms of the above such as an enantiomeric mixture that is partially racemic in that it comprises at least 51%, at least 55%, at least 60%, at least 65%, at least 70%, at least 75%, at least 80%, at least 85%, at least 90%, at least 95%, at least 96%, at least 97%, at least 98%, at least 99% of one enantiomeric form over the other. The aforementioned fatty acid compounds as physiologically acceptable esters such as polyhydric alcohol esters, e.g., glycerol esters, are specifically included. Also, specifically included are methods of reducing or eliminating metabolic caloric content of a food composition comprising an edible fat where at least a portion of the edible fat of the food composition is replaced by R,R,S-phytanic acid, R,R,R-phytanic acid, R,R,S-pristanic acid or R,R,R-pristanic acid or combinations thereof and including physiologically acceptable esters thereof and metabolic precursors thereof such as, for example, R,R,R-phytol as a metabolic precursor to R,R,R-phytanic acid and subsequently R,R,R-pristanic acid, and R,R,S-phytol as a metabolic precursor to R,R,S-phytanic acid and subsequently R,R,S-pristanic acid.

Preparation of the Fatty Acid Compounds of the Invention

The fatty acid compounds of the present invention may be prepared by methods that include steps that are individually known in the art but are not known in the art in combination for the preparation of the fatty acid compounds of the invention. Some of the compounds of the invention may be prepared by known polymerization techniques, which are chosen based on the particular compound of the invention being made.

Fatty Acid Compounds with Quaternary Center at Alpha Carbon

The following preparative scheme is for the preparation of fatty acid compounds wherein the alpha carbon has a quaternary center. The starting material may be a known fatty acid having a single hydrogen atom on the alpha carbon. The known fatty acid is usually a racemic mixture. The known fatty acid employed as the starting material may be synthetic or natural, saturated or unsaturated, with straight or branched chains. Examples of such fatty acids are caproic, caprylic, pelargonic, capric, undecanoic, lauric, myristic, myristoleic, palmitic, margaric, stearic, arachidic, behenic, behenolic, erucic, erucidic (brassic), heptadecanoic, lignoceric, cerotic, montanic, mellissic, palmitoleic (zoomaric), palmitolic, ricinoleic, oleic, vaccenic, linoleic, linolenic, eleostearic, arachidonic, nervonic, eicosapentaenoic, decosatetraenoic, decosapentaenoic, decosahexaenoic, and the like acids.

The carboxyl group of the known fatty acid is converted to an amide functionality. Typically, amides may be synthesized from the corresponding carboxylic acid by treatment of the free carboxylic acid, an ester thereof, an acid halide thereof, an anhydride thereof and so forth with ammonia. The specific reaction conditions will not be discussed herein. See, for example, Org. Prep. Proc. Int., 14, 396-(1982). The resulting amide is then treated to convert the amide to a nitrile or cyano group. One approach involves dehydration of the amide with a suitable dehydration agent and dehydration conditions. The dehydration agent may be, for example, phosphorous oxychloride, and the like in an anhydrous organic solvent such as, e.g., a halogenated hydrocarbon (dichloromethane, carbon tetrachloride, chloroform, etc.), and so forth. In some embodiments, the temperature for the dehydration is about 60° C. to about 100° C. An alkyl substituent is then introduced on the alpha carbon by an alkylation reaction using the desired alkylating agent in the form of an alkyl halide (alkyl iodide, alkyl bromide, alkyl chloride), alkyl silyl chloride and the like. The alkylation reaction is usually carried out in the presence of a base such as, for example, sodium azide, KNH₂, sodium, n-butyl lithium, and the like. Usually, the temperature for the alkylation is about 0° C. to about −100° C. for a period of about 5 minutes to about 24 hours. Then, the cyano group is hydrolyzed to give the carboxyl group. The hydrolysis may be carried out under acid or basic conditions. Usually, relatively strong acidic or basic conditions are employed. The acid may be, for example, a mineral acid such as, e.g., hydrochloric acid, sulfuric acid, phosphoric acid, and the like, an organic acid such as, e.g., trifluoroacetic acid, o-chlorobenzoic acid. The acid strength depends on the acid employed. The base may be, for example, a metal hydroxide such as, e.g., sodium hydroxide, potassium hydroxide, etc., and so forth. The strength of the base depends on the nature of the base. In general, hydrolysis conditions are well known in the art and will not be discussed in detail. The hydrolysis is carried out in an aqueous medium usually under reflux conditions.

The resulting fatty acid compounds may be purified by known techniques such as, for example, extraction, column chromatography, thin-layer chromatography, thick-layer chromatography, preparative low or high-pressure liquid chromatography or a combination of these procedures.

The fatty acid compounds may also be prepared by conversion of a terminal alkene to a nitrile or oxidation of a terminal alkene to a carboxylic acid. In either case, the aforementioned processes can then be carried out.

Fatty Acid Compounds that are Substantially S-Enantiomers

Fatty acid compounds that are S-enantiomers substantially free from R-enantiomers may be prepared by the following illustrative procedures. In one approach, the starting material may be a known fatty acid. The known fatty acid employed as the starting material may be synthetic or natural, saturated or unsaturated, with straight or branched chains. Examples of such fatty acids are caproic, caprylic, pelargonic, capric, undecanoic, lauric, myristic, myristoleic, palmitic, margaric, stearic, arachidic, behenic, behenolic, erucic, erucidic (brassic), heptadecanoic, lignoceric, cerotic, montanic, mellissic, palmitoleic (zoomaric), palmitolic, ricinoleic, oleic, vaccenic, linoleic, linolenic, eleostearic, arachidonic, nervonic, eicosapentaenoic, decosatetraenoic, decosapentaenoic, decosahexaenoic, and the like acids.

The carboxyl group of the known fatty acid is converted to a nitrile functionality. Typically, nitriles may be synthesized from the corresponding carboxylic acid by a multistep procedure beginning with the treatment of the free carboxylic acid, an ester thereof, an acid halide thereof, an anhydride thereof and so forth with a reducing agent such as, for example, a metal hydride, e.g., LiAlH₄. The specific reaction conditions will not be discussed herein. See, for example, Org. React (1951) 6 469.

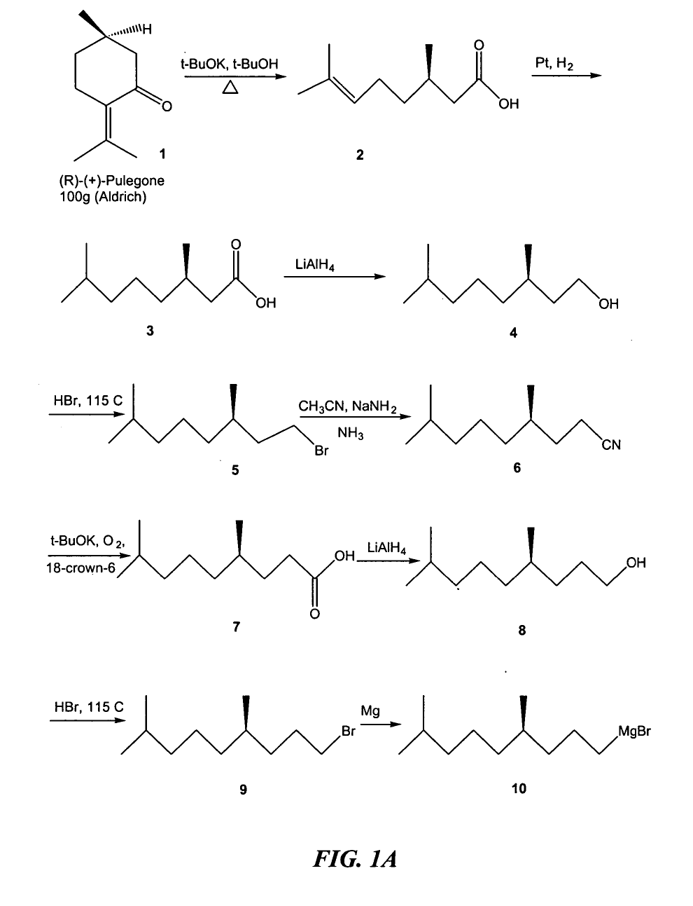

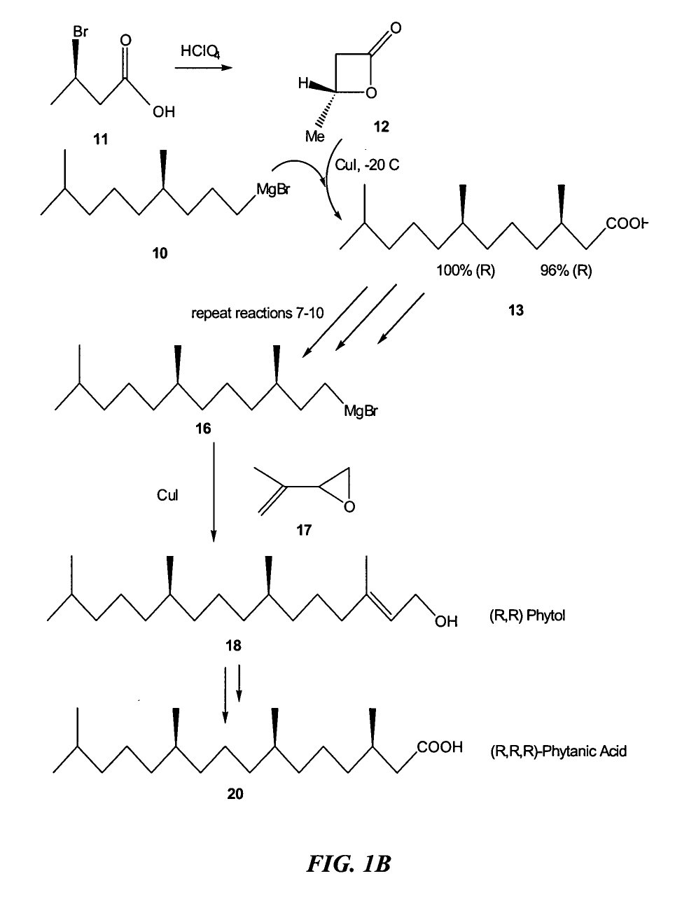

The resulting alcohol is then treated to convert the alcohol to a halide group. One approach involves reaction with hydrogen bromide at 115° C. The halogenation agent may be, for example, hydrogen chloride, hydrogen iodide, thionyl chloride and the like. A nitrile substituent is then introduced by reaction of the bromide with, for example, acetonitrile and NaNH₂ in ammonia. Then, the cyano group is hydrolyzed to give the carboxyl group. The hydrolysis may be carried out under acid or basic conditions. Usually, relatively strong acidic or basic conditions are employed. The acid may be, for example, a mineral acid such as, e.g., hydrochloric acid, sulfuric acid, phosphoric acid, and the like, an organic acid such as, e.g., trifluoroacetic acid, o-chlorobenzoic acid. In general, hydrolysis conditions are well known in the art and will not be discussed in detail. The hydrolysis is carried out in an aqueous medium usually under reflux conditions. Reduction followed by bromination and cyanation adds one carbon fragment to the molecule in question. An example of adding a chiral three-carbon fragment is illustrated in FIGS. 1A-1B. From the fatty acid derived bromide in question, a Grignard reagent is formed by reaction with magnesium. The lactone of (S)-(+)-3-bromobutyric acid is formed by reaction with perchloric acid. In the presence of cuprous iodide at −20° C., the lactone of (S)-(+)-3-bromobutyric acid is reacted with the above Grignard reagent derived from starting fatty acid to form in very good yield, stereospecifically, the R enantiomer of the methyl substituted fatty acid at the 3-position carbon. This same methodology can be repeated to increase chain length. Also, by starting with the lactone of (R)-(+)-3-bromobutyric acid, the resulting S enantiomer of the methyl-substituted fatty acid at the 3-position carbon can be obtained. Mixtures of the R-enantiomers and the S-enantiomers of desired distribution of enantiomers may be prepared by starting with appropriate mixtures of the above materials.

A particular example of the above procedure is depicted in FIGS. 1A-1B. (R,R,R) Phytanic Acid is the desired, physiologically relevant fatty acid being synthesized in a stereo-controlled fashion in the procedure depicted. In FIGS. 1A-1B, t-bu is t-butyl.

In another manner, compounds with the S enantiomer substantially free of the R enantiomer may be produced by incubating the racemic compound with the fungus Verticillium lecanii. This approach is illustrated by the following example preparation: a 1 mL inoculate of V. lecanii is grown to the end of log phase (at 96 hours) in 100 mL of Dextrose-Peptone medium. The cultures are harvested by centrifugation at 1000×g, washed 3 times with sterile distilled water and then resuspended in 100 mL of sterile Sorensen's phosphate buffer pH 6.0 at a cell density of 20 mg/mL dry cell weight. The compound to be converted from racemate to mostly S enantiomer is added to a final concentration of 250 mg/mL and the pH readjusted to 6.0. The flasks containing V. lecanii and compound of interest are incubated at 24° C. on a shaker rotating at 150 rpm for up to 6 days. Subsequently, the cultures are centrifuged in a suitable vessel at 2000×g for 5 minutes. The compound of interest can then be purified from the supernatant.

While the above process is exemplified for small amounts of compound, the process can be easily be performed at a larger scale by those skilled in the art.

Compositions Comprising Fatty Acid Compounds of the Invention

The fatty acid compounds of the present invention, or physiologically acceptable esters thereof, may be admixed with known edible fats to provide characteristics such as flavor, beneficial effects from components of some fatty acids such as omega-3 fatty acids and omega-6 fatty acids, and so forth. The amount of known edible fat or known edible fats mixed with a fatty acid compound of the invention depends on the nature of the edible fat, the desired characteristic to be imparted, and so forth. The amount of known edible fat in a composition in admixture with a fatty acid compound of the invention may be about 0.1 to about 99.9%, about 1 to about 99%, about 2 to about 98%, about 3 to about 97%, about 4 to about 96%, about 5 to about 95%, about 10 to about 90%, about 20 to about 80%, about 30 to about 70%, about 40 to about 60%, about 50 to about 50%, and so forth. For example, a fatty acid compound of the invention may be mixed with olive oil in an amount within all of the above ranges.

Other known edible fats can be synthetic or derived from animal or vegetable sources, or combinations of these. The known edible fats may be those derived from non-hydrogenated, partially hydrogenated or fully hydrogenated soybean, safflower, sunflower, sesame, peanut, corn, rice bran, canola, babassu nut, coconut, palm, palm kernel, lupin, nasturtium seed, mustard seed, cottonseed, low erucic rapeseed, butter or marine oils, or plant waxes such as jojoba. Known edible fats also include edible fat replacements, including, but not limited to, sugar esters, neoalkyl esters, polyglycerol esters, malonate esters, propoxylated glycerols, retrofats, silicone oils/siloxanes, carboxy/carboxylates, and the like. The aforementioned combination of known edible fats, shortening and oil products include, but are not limited to shortenings, margarines, spreads, butter blends, lards, cooking and frying oils, salad oils, popcorn oils, salad dressings, mayonnaise, and other edible oils.

Advantageously, the known edible fats utilized may be selected to provide the desired fatty characteristics to a compound of the invention, if necessary. The aforementioned blends or combinations can be selected for a desired rheology, melt profile, and mouth feel. This is especially desirable in the case of margarine substitutes, cookie fillings, whipped toppings, etc. Among the esters for many applications are those with melting points below about 98° F. because these materials melt in the mouth providing the organoleptic sensation of natural fats and oils. For some products, relatively sharp melting points, for instance in the range of from about 90° F. to about 98° F., are desired because they provide a cooling sensation and a meltdown equivalent to high quality, solid, natural fats. It is, however, to be noted that one of the advantages of the present fatty acid compounds is that they have, in may cases, the taste and feel of known edible fats and, thus, admixture may not be necessary.

In a particular example, the fatty acid compounds of the invention can be employed in margarine substitutes, which can be either soft or hard. Margarines are generally sold as one of two principal types: namely, (1) hard or stick margarine and (2) soft or tub margarine. All of these products contain liquid and hard stock components that can be replaced by compounds of the invention. It is an advantage of the present invention that, by eliminating some or all of the hard stock of conventional margarines, higher ratios of polyunsaturated to saturated fatty acids and lesser amounts of trans isomers can be achieved in high quality margarine products.

The fatty acid compounds of the invention, or physiologically acceptable esters thereof, either individually or in combination with other edible fats may be employed as the fat ingredient in a food composition comprising a non-fat ingredient and a fat ingredient. The amount of the fatty acid compound in the food composition is determined by the nature of the non-fat ingredient, the intended use of the food composition, and so forth. The amount of the fatty acid compound of the invention in the food composition is about 1% to about 100%, about 10% to about 99%, about 20% to about 95%, about 30% to about 80%, about 40 to about 60%, and so forth.

The non-fat ingredient may be any edible material, whether or not intended for nutrition. The non-fat ingredient may be a protein (from animal or vegetable sources) or a carbohydrate (starches, sugars, celluloses) or mixtures thereof. The non-fat ingredient may be an additive such as an antioxidant for fats or oils, an antispatter agent, an emulsifier, an edible lipid (triglycerides), a vitamin, a thickener, a preservative, a colorant, a flavoring agent, a fragrance, a sugar substitute, water, a spice, or other minor functional ingredient and so forth, or mixtures thereof.

Ordinary food compositions containing from about 1 to 100% of the usual fat component in the form of a fatty acid compound of the invention may be prepared in any conventional manner of mixing, blending, cooking until the food product is adjudged “done” in the known sense, and so forth, and combinations thereof. The aforementioned range covers partial to full substitution of ordinary fat-type ingredients with the compounds of this invention. At the 1% level there is a partial substitution, while at the 100% level there is a full substitution.

The fatty acid compounds of the present invention, or blends or esters thereof, are useful in a wide variety of food and beverage products. For example, they can be used in the production of baked goods in any form, such as mixes, shelf-stable baked goods, and frozen baked goods. Possible applications include, but are not limited to, cakes, brownies, muffins, bar cookies, wafers, biscuits, pastries, pies, pie crusts, and cookies, including sandwich cookies and chocolate chip cookies, storage-stable dual-textured cookies, and so forth. The baked goods can contain fruit, cream, or other fillings. Other baked good uses include breads and rolls, crackers, pretzels, pancakes, waffles, ice cream cones and cups, yeast-raised baked goods, pizzas and pizza crusts, and baked farinaceous snack foods, and other baked salted snacks.

Representative examples of food products that can contain the fatty acid compounds of the invention in full or partial replacement of natural fat are: frozen desserts, e.g., sherbet, ice cream, ices, or milk shakes; puddings and pie fillings; margarine substitutes or blends; flavored bread or biscuit spreads; mayonnaise; salad dressing, both emulsified and non-emulsified; filled dairy products such as filled cream or filled milk; dairy or non-dairy cheese spreads; peanut butter; egg substitutes; coffee lighteners, liquid and dried; flavored dips; frying fats and oils; reformed and comminuted meats; meat substitutes or extenders; whipped toppings; compound coatings; frostings and fillings; cocoa butter replacements or blends; candy, especially fatty candies such as containing peanut butter or chocolate; chewing gum; bakery products, e.g., cakes, breads, rolls, pastries, cookies, biscuits, or savory crackers; mixes or ingredient premixes for any of these; as well as flavor, nutrient, drug, or functional additive delivery systems.

The present food compositions or the present fatty acid compounds themselves may be fortified with vitamins and minerals, particularly the fat-soluble vitamins. The fat-soluble vitamins include vitamin A, vitamin D, vitamin E, and vitamin K. Vitamin A is a fat-soluble alcohol. Natural vitamin A is usually found esterified with a fatty acid; metabolically active forms of vitamin A also include the corresponding aldehyde and acid. Vitamin D is a fat-soluble vitamin well known for use in the treatment and prevention of rickets and other skeletal disorders. “Vitamin D” comprises sterols, and there are at least 11 sterols with vitamin D-type activity. Vitamin E (tocopherol) is a third fat-soluble vitamin that can be used in the present invention. Four different tocopherols have been identified (alpha, beta, gamma and delta), all of which are oily, yellow liquids, insoluble in water but soluble in fats and oils. Vitamin K exists in at least three forms, all belonging to the group of chemical compounds known as quinones. The naturally occurring fat-soluble vitamins are K₁ (phylloquinone), K₂ (menaquinone), and K₃ (menadione). The amount of the fat-soluble vitamins employed herein to fortify the present low calorie fat materials can vary. If desired, the fat materials can be fortified with a recommended daily allowance (RDA), or increment or multiple of an RDA, of any of the fat-soluble vitamins or combinations thereof.

Vitamins that are non-soluble in fat can similarly be included in the present non-digestible solid fat materials. Among these vitamins are the vitamin B complex vitamins, vitamin C, vitamin G, vitamin H, and vitamin P. The minerals include the wide variety of minerals known to be useful in the diet, such as calcium, magnesium, and zinc. Any combination of vitamins and minerals can be used in the present low-calorie fat materials.

The present fatty acid compounds, including esters and blends thereof, are particularly useful in combination with particular classes of food and beverage ingredients. For example, an extra calorie reduction benefit is achieved when the fat materials are used with non-caloric or reduced calorie sweeteners alone or in combination with bulking agents. Non-caloric or reduced calorie sweeteners include, but are not limited to, aspartame; saccharin; alitame, thaumatin; dihydrochalcones; cyclamates; steviosides; glycyrrhizins, synthetic alkoxy aromatics, such as Dulcin and P-4000; sucrolose, suosan; miraculin; monollin; sorbitol; xylitol; talin; cyclohexyl sulfamates; substituted imidazolines; synthetic sulfamic acids such as acesulfame, acesulfam-K and n-substituted sulfamic acids; oximes such as perilartine; rebaudioside-A; peptides such as aspartyl malonates and succanilic acids; dipeptides; amino acid based sweeteners such as gem-diaminoalkanes, meta-aminobenzoic acid, L-aminodicarboxylic acid alkanes, and amides of certain alpha-aminodicarboxylic acids and gem-diamines; and 3-hydroxy-4-alkyloxyphenyl aliphatic carboxylates or heterocyclic aromatic carboxylates.

Bulking or bodying agents are useful in combination with the present fatty acid compounds, and blends and esters thereof, in many food compositions. The bulking agents can be non-digestible carbohydrates, for example, polydextrose and cellulose or cellulose derivatives, such as carboxymethylcellulose, carboxyethylcellulose, hydroxypropylcellulose, methylcellulose and microcrystalline cellulose. Other suitable bulking agents include gums (hydrocolloids), starches, dextrins, fermented whey, tofu, maltodextrins, polyols, including sugar alcohols, e.g., sorbitol and mannitol, and carbohydrates, e.g., lactose.

Similarly, food and beverage compositions can be made that combine the present fatty acid compounds with dietary fibers to achieve the combined benefits of each. By “dietary fiber” is meant complex carbohydrates resistant to digestion by mammalian enzymes, such as the carbohydrates found in plant cell walls and seaweed, and those produced by microbial fermentation. Examples of these complex carbohydrates are brans, celluloses, hemicelluloses, pectins, gums and mucilages, seaweed extract, and biosynthetic gums. Sources of the cellulosic fiber include vegetables, fruits, seeds, cereals, and manmade fibers (for example, by bacterial synthesis). Commercial fibers such as purified plant cellulose, or cellulose flour, can also be used. Naturally occurring fibers include fiber from whole citrus peel, citrus albedo, sugar beets, citrus pulp and vesicle solids, apples, apricots, and watermelon rinds.

The dietary fibers may be in a crude or purified form. The dietary fiber used may be of a single type (e.g., cellulose), a composite dietary fiber (e.g., citrus albedo fiber containing cellulose and pectin), or some combination of fibers (e.g., cellulose and a gum). The fibers can be processed by methods known to the art.

Of course, judgment must be exercised to make use of the fatty acid compounds of the present invention and combinations thereof with other food ingredients. For example, a combination of sweetener and a present fatty acid compound would not be used where the specific benefits of the two are not desired. The fatty acid compounds of the invention, and blends and esters thereof, and other combinations are used where appropriate, and in appropriate amounts.

Many benefits are obtained from the use of the present compounds in food and beverage compositions, either when used alone or in combination with edible oils and/or other ingredients discussed above. A primary benefit is the calorie reduction achieved as a result of total or partial fat replacement. This calorie reduction can be increased by using combinations of the present compounds with reduced calorie sweeteners, bulking agents, and the like. Another benefit that follows from this use is a decrease in the total amount of fats in the diet. Foods or beverages made with the present compounds will also contain less cholesterol, and the ingestion of these foods can lead to reduced serum cholesterol and thus reduced risk of heart disease.

Dietary foods can be made with the fatty acid compounds of the invention, and blends and esters thereof, to meet special dietary needs, for example, of persons who are obese, diabetic, atherosclerotic, or hypercholesterolemic. The present compounds can be a major part of a low-fat, low calorie, low-cholesterol diet, and they can be used alone or in combination with drug therapy or other therapy. Combinations of food or beverage products made with the present fatty acid compounds can be used as part of a total dietary management regimen, based on one or more of these products, containing the fat materials alone or in combination with one or more of the above-mentioned ingredients, to provide one or more of the above-mentioned benefits.

The present fatty acid compounds, and blends and esters thereof, do not have some of the deleterious effects realized from some of the known fat substitutes such as, for example, anal leakage, decrease in the availability of the fat-soluble vitamins A.D, E, and K, diarrhea, loose stools, gas and abdominal cramping, and so forth.

In addition to food compositions, the compounds of the present invention can also be used in formulating lubricants, skin creams, pharmaceutical ointments, and the like.

In addition to the above, the fatty acid compounds of the present invention may have a preventative or curative effect for various diseases or illnesses. Such as, by way of illustration and not limitation, inflammation, cystic fibrosis, essential fatty acid syndrome, dementia, neoplastic disease, endocrinologic diseases and neurodegenerative diseases, and so forth. Dementia includes Alzheimer's disease, Parkinson's disease, Charcot-Marie-Tooth Disease, Amyothrophic lateral sclerosis, dementia with lewy bodies, and so forth. Inflammation includes arthritis and the like, Hepatitis and the like, inflammatory bowel disease, colitis and the like, Crohn's disease, Systemic Lupus Erymathosis (SLE), and so forth. Neoplastic disease includes cancers and adenocarcinomas, for instance, gastrointestinal cancers including colon cancer, rectal cancer, breast cancer, ovarian, cancer, endometrial cancer, thyroid cancer, lung cancer, leukemia, lymphoma, cancer of the larynx, cervical cancer, prostate cancer, testicular cancer, bladder cancer, kidney cancer, pancreatic cancer, myeloma, squamous cell carcinoma, brain tumors, and lipoma.

The invention is further illustrated by reference to the following examples describing the preparation of some of the compositions of the present invention, as well as their utility. It will be apparent to those skilled in the art that many modifications, both to materials and methods, may be practiced without departing from the purpose and interest of this invention.

It should be understood that the above description is intended to illustrate and not limit the scope of the invention. Other aspects, advantages and modifications within the scope of the invention will be apparent to those skilled in the art to which the invention pertains. The following examples are put forth so as to provide those of ordinary skill in the art with examples of how to make and use the methods and products of the invention, and are not intended to limit the scope of what the inventors regard as their invention.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events that is logically possible, as well as the recited order of events.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method of reducing or eliminating metabolic caloric content of a food composition comprising an edible fat, said method comprising substituting, for at least a portion of the edible fat of said food composition, a compound that is a fatty acid whose yield energy from beta-oxidation is sufficiently low that said compound is rendered metabolically low caloric or non-caloric, or a physiologically acceptable ester thereof or a metabolic precursor thereof.

2. A method according to claim 1 wherein said compound has from 6 to about 30 carbon atoms and from 0 to about 10 unsaturations.

3. A method according to claim 1 wherein said compound has at least one unsaturation that is a double bond.

4. A method according to claim 1 wherein said compound is an ester of a polyhydric alcohol.

5. A method according to claim 1 wherein said compound has the formula:
A-(metabolic blocker)-CO₂H
wherein said metabolic blocker renders the yield energy from beta-oxidation of said compound sufficiently low that said compound is rendered metabolically low caloric or non-caloric and wherein A is alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations, or alkyne having from 2 to about 30 carbon atoms, or substituted alkyne having 2 to about 30 carbon atoms and having from 1 to about 10 unsaturations, or a physiologically acceptable ester thereof or a metabolic precursor thereof.

6. A method according to claim 5 wherein said metabolic blocker has the formula:
—C(X)(D)-,
wherein X is alkyl of 1 to about 10 carbon atoms and D is hydrogen or alkyl of 1 to about 10 carbon atoms and wherein, when D is hydrogen, said metabolic blocker is predominantly an S-enantiomer or is an S-enantiomer substantially free from R-enantiomer and wherein A is alkyl of 1 to about 30 carbon atoms, substituted alkyl of 1 to about 30 carbon atoms, alkene of 2 to about 30 carbon atoms and having from 1 to about 10 unsaturations, or substituted alkene having from 2 to about 30 carbon atoms and having from 1 to about 10 unsaturations.

7. A method according to claim 6 wherein X is methyl and D is hydrogen.

8. A method according to claim 6 wherein X is methyl and D is methyl.

9. A method according to claim 5 wherein said metabolic blocker has the formula:
—(CH₂(CH₂)ₐCH(Y)(CH₂(CH₂)_{b}CH (Y))ₘ(CH₂(CH₂)_{c}CH(V)—
wherein m is 0 to 3 and wherein V and Y are independently alkyl having from 1 to about 10 carbon atoms and
(i) wherein when the carbon atom comprising the V group is predominantly an S-enantiomer or is an S-enantiomer substantially free from R-enantiomer, the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and a, b and c are independently an integer of 1 to 5 or
(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and a, b and c are independently an integer of 1 to 5 and the b or c before the carbon atom that is the S-enantiomer is an integer of 1 or 3 or
(iii) wherein, when the carbon atom comprising the V group is predominantly an R-enantiomer or is an R-enantiomer substantially free from S-enantiomer and the carbon atoms comprising the Y-groups are independently predominantly an R-enantiomer or are R-enantiomers substantially free from S-enantiomers, at least one of a, b and c is an integer of 1 or 3 and the others are an integer of 1 to 5 and wherein the carbon atoms not comprising the V group or the Y groups may be substituted with one or more substituents and
wherein A is alkyl of 1 to about 20 carbon atoms, substituted alkyl of 1 to about 20 carbon atoms, alkene of 2 to about 20 carbon atoms and having from 1 to about 5 unsaturations, or substituted alkene having from 2 to about 20 carbon atoms and having from 1 to about 5 unsaturations.

10. A method according to claim 9 wherein V is methyl and Y is methyl.

11. A method according to claim 5 wherein A is selected from the group consisting of CₙH₂ₙ₊₁, CₙH₂ₙ₋₁, CₙH₂ₙ₋₃, and CₙH₂ₙ₋₅ wherein n is 4 to 20 and wherein 1 to 5 carbon atoms are optionally substituted.

12. A method according to claim 5 wherein said compound is an ester of a polyhydric alcohol.

13. A method according to claim 5 wherein said compound is an ester of a triglyceride.

14. A method according to claim 1 wherein said compound comprises about 1% to about 100% of said edible fat.

15. A method according to claim 1 wherein said food composition comprises a protein or a carbohydrate or both.

16. A method according to claim 1 wherein said compound is a fatty acid substantially incapable of beta-oxidation, or a physiologically acceptable ester thereof or a metabolic precursor thereof.

17. A method according to claim 1 wherein said compound is a fatty acid having a yield energy from beta-oxidation that is low enough to render said fatty acid metabolically low caloric, or a physiologically acceptable ester thereof or a metabolic precursor thereof.

18. A method according to claim 1 wherein said compound is selected from the group consisting of R,R,S-pristanic acid, R,R,R-pristanic acid, R,R,R-phytanic acid, R,R,S-phytanic acid, R,R,R-phytol and R,R,S-phytol and physiologically acceptable esters thereof.

19. A method for rendering an organic acid compound metabolically low caloric or non-caloric said method comprising introducing into said compound one or more substituents at a carbon atom that is alpha to a carboxyl group of said organic acid compound wherein said substituent renders the yield energy from beta-oxidation of said organic acid compound sufficiently low that said compound is rendered metabolically low caloric or non-caloric.

20. A method according to claim 19 wherein said organic acid compound has from 4 to about 30 carbon atoms and from 0 to about 10 unsaturations.

21. A method according to claim 19 wherein one substituent is introduced and wherein said substituent is alkyl of 1 to about 10 carbon atoms and said fatty acid compound is predominantly an S-enantiomer or is an S-enantiomer substantially free from R-enantiomer.

22. A method according to claim 19 wherein two substituents are introduced and wherein said substituents are independently alkyl of 1 to about 10 carbon atoms.

23. A method according to claim 19 wherein said substituent is methyl.

24. A method according to claim 19 wherein said fatty acid compound has the formula selected from the group consisting of CₙH₂ₙ₊₁COOH, CₙH₂ₙ₋₁COOH, CₙH₂ₙ₋₃COOH and CₙH₂ₙ₋₅COOH wherein n is 5 to 20 and wherein 1 to 5 carbon atoms are optionally substituted.

25. A compound of the formula:
A′-(metabolic blocker)-CO₂H
wherein said metabolic blocker renders said compound substantially incapable of beta-oxidation and wherein A′ is alkyl of about 4 to about 30 carbon atoms, substituted alkyl of 1 to about 30 carbon atoms, alkene of about 4 to about 30 carbon atoms and having from 1 to about 10 unsaturations, or substituted alkene having from about 4 to about 30 carbon atoms and having from 1 to about 10 unsaturations, or alkyne having from 2 to about 30 carbon atoms, or substituted alkyne having 2 to about 30 carbon atoms and having from 1 to about 10 unsaturations, or a physiologically acceptable ester thereof or a metabolic precursor thereof.

26. A compound according to claim 25 wherein said metabolic blocker has the formula:
—C(X¹)(D)-,
wherein X¹ is alkyl of 1 to about 10 carbon atoms and D is hydrogen or alkyl of 1 to about 10 carbon atoms and wherein, when D is hydrogen, said metabolic blocker is predominantly an S-enantiomer or is an S-enantiomer substantially free from R-enantiomer and wherein A′ is alkyl of about 4 to about 20 carbon atoms, substituted alkyl of about 4 to about 20 carbon atoms, alkene of about 4 to about 20 carbon atoms and having from 1 to about 5 unsaturations, or substituted alkene having from about 4 to about 20 carbon atoms and having from 1 to about 5 unsaturations.

27. A compound according to claim 25 wherein A′ is selected from the group consisting of CₙH₂ₙ₊₁, CₙH₂ₙ₋₁, CₙH₂ₙ₋₃, and CₙH₂ₙ₋₅ wherein n is 4 to 20 and wherein 1 to 5 carbon atoms are optionally substituted.

28. A compound according to claim 25 which is an ester of a triglyceride.

29. A food composition comprising an edible fat and a compound according to claim 25.

30. A food composition comprising a non-fat ingredient and a fat ingredient at least a portion of which is a compound according to claim 25.

31. A food composition according to claim 30 wherein said fat ingredient comprises about 1% to about 100% of said compound.

32. A food composition according to claim 30 wherein said non-fat ingredient comprises a protein or a carbohydrate or both.

33. A compound according to claim 25 wherein said metabolic blocker has the formula:
—(CH₂(CH₂)ₐCH(Y)(CH₂(CH₂)_{b}CH(Y))ₘ(CH₂(CH₂)_{c}CH(V)—
wherein m is 0 to 3 and wherein V and Y are independently alkyl having from 1 to about 10 carbon atoms and
(i) wherein when the carbon atom comprising the V group is predominantly an S-enantiomer or is an S-enantiomer substantially free from R-enantiomer, the carbon atoms comprising the Y groups are independently R-enantiomers substantially free from S-enantiomers or S-enantiomers substantially free from R-enantiomers or one or more of the carbon atoms comprising the Y groups may be a combination of S-enantiomers and R-enantiomers (thereby rendering the fatty acid compound wholly or partially racemic with respect to such carbon atom(s)) and a, b and c are independently an integer of 1 to 5 or
(ii) wherein, when the carbon atom comprising the V group is predominantly an R-enantiomer or is an R-enantiomer substantially free from S-enantiomer and one of the carbon atoms comprising the Y groups is predominantly an S-enantiomer or is an S-enantiomer substantially free from R-enantiomer and the other carbon atoms comprising the Y-groups are independently R-enantiomers substantially free from S-enantiomers or S-enantiomers substantially free from R-enantiomers or one or more of the carbon atoms after (with respect to right to left in the above formula) the carbon atom that is the S-enantiomer may be a combination of S-enantiomers and R-enantiomers (thereby rendering the fatty acid compound wholly or partially racemic with respect to such carbon atom(s)) and a, b and c are independently an integer of 1 to 5 and the b or c before the carbon atom that is the S-enantiomer is an integer of 1 or 3 or
(iii) wherein, when the carbon atom comprising the V group is predominantly an R-enantiomer or is an R-enantiomer substantially free from S-enantiomer and the carbon atoms comprising the Y-groups are independently predominantly an R-enantiomer or are R-enantiomers substantially free from S-enantiomers, at least one of a, b or c is an integer of 1 or 3 and the others are an integer of 1 to 5 and wherein the carbon atoms not comprising the V group or the Y groups may be substituted with one or more substituents and
wherein A′ is alkyl of 1 to about 20 carbon atoms, substituted alkyl of 1 to about 20 carbon atoms, alkene of 2 to about 20 carbon atoms and having from 1 to about 5 unsaturations, or substituted alkene having from 2 to about 20 carbon atoms and having from 1 to about 5 unsaturations.

34. A compound according to claim 33 wherein the metabolic blocker has the formula:
—CH₂CH₂CH(Y)(CH₂CH₂CH(Y))ₘCH₂CH₂CH₂CH(V)—.

35. A compound according to claim 33 wherein the metabolic blocker has the formula:
—CH₂CH₂CH(Y)(CH₂CH₂CH₂CH₂CH(Y))ₘCH₂CH₂CH₂CH(V)—

36. A compound according to claim 33 which is a physiologically acceptable ester of a compound selected from the group consisting of R,R,S-pristanic acid, R,R,R-pristanic acid, R,R,R-phytanic acid, R,R,S-phytanic acid, R,R,R-phytol and R,R,S-phytol.

37. A compound of the formula:
A″-C(X²)(D¹)-CO₂H
wherein X² is alkyl of 1 to about 10 carbon atoms and D¹ is alkyl of 1 to about 10 carbon atoms and wherein A″ is alkyl of about 4 to about 30 carbon atoms, substituted alkyl of 4 to about 30 carbon atoms, alkene of about 4 to about 30 carbon atoms and having from 1 to about 10 unsaturations, or substituted alkene having from about 4 to about 30 carbon atoms and having from 1 to about 10 unsaturations, or alkyne having from 4 to about 30 carbon atoms, or substituted alkyne having 4 to about 30 carbon atoms and having from 1 to about 10 unsaturations, or a physiologically acceptable ester thereof or a metabolic precursor thereof.

38. A compound according to claim 37 wherein A″ is alkyl of about 4 to about 20 carbon atoms, substituted alkyl of about 4 to about 20 carbon atoms, alkene of about 4 to about 20 carbon atoms and having from 1 to about 5 unsaturations, or substituted alkene having from about 4 to about 20 carbon atoms and having from 1 to about 5 unsaturations.

39. A compound according to claim 37 wherein A″ is selected from the group consisting of CₙH₂ₙ₊₁, CₙH₂ₙ₋₁, CₙH₂ₙ₋₃, and CₙH₂ₙ₋₅, wherein n is 4 to 20 and wherein 1 to 5 carbon atoms are optionally substituted.

40. A compound according to claim 37 wherein said compound is an ester of a triglyceride.

41. A food composition comprising an edible fat and a compound according to claim 37.

42. A food composition comprising a non-fat ingredient and a fat ingredient at least a portion of which is a compound according to claim 37.

43. A food composition according to claim 42 wherein said fat ingredient comprises about 1% to about 100% of said compound.

44. A food composition according to claim 43 wherein said non-fat ingredient comprises a protein or a carbohydrate or both.

45. A physiologically acceptable ester of a compound, which is a fatty acid whose yield energy from beta-oxidation is sufficiently low that said compound is rendered metabolically low caloric or non-caloric.

46. A compound according to claim 45 having from 4 to about 30 carbon atoms and from 0 to about 10 unsaturations.

47. A compound according to claim 45, which has at least one unsaturation that is a double bond.

48. A compound according to claim 45 wherein said ester is an ester of a polyhydric alcohol.
